(19) United States
(12) Patent Application Publication
(10) Pub. No.: US 2024/0181036 A1
(43) Pub. Date: Jun. 6, 2024

(54) TRANSDERMAL ACTIVE AGENT DELIVERY DEVICES HAVING CORONAVIRUS VACCINE COATED MICRO-PROTRUSIONS

(71) Applicant: Emergex USA Corporation, Doylestown, PA (US)

(72) Inventors: Mahmoud AMERI, Fremont, CA (US); Hayley LEWIS, Redwood City, CA (US)

(73) Assignee: Emergex USA Corporation, Doylestown, PA (US)

(21) Appl. No.: 17/996,882

(22) PCT Filed: Apr. 22, 2021
(86) PCT No.: PCT/US2021/028715

§ 371 Date: Oct. 21, 2022

Related U.S. Application Data
(60) Provisional application No. 63/013,809, filed on Apr. 22, 2020.

(51) Int. Cl. A61K 39/145; A61K 9/00; A61K 47/26; A61P 31/16; C07K 14/11

(52) U.S. Cl. CPC A61K 39/145; A61K 9/0021; A61K 47/26; A61P 31/16; C07K 14/11; A61K 2039/54; C12N 2760/16034; C12N 2770/20034

(57) ABSTRACT

Disclosed herein are systems and methods for the transdermal or intracutaneous delivery of vaccines, and more particularly to the delivery of vaccines that produce coronavirus or other virus specific antibodies in the serum of vaccinated mammals, including to prevent COVID-19.

### CROSS REFERENCE TO RELATED APPLICATION

This application claims benefit of U.S. Provisional Patent Application No. 63/013,809 filed on Apr. 22, 2020; which is incorporated herein by reference in its entirety to the full extent permitted by law.

### FIELD

The present invention relates to the field of transdermal or intracutaneous delivery of vaccines, and more particularly to the delivery of vaccines that produce coronavirus or other virus specific antibodies in the serum of vaccinated mammals.

### BACKGROUND

The influenza vaccine is a yearly vaccine that protects people from getting the flu, a viral respiratory illness that spreads easily. The flu vaccine is typically administered by injection or intranasal spray. With the recent coronavirus pandemic, researchers are actively investigating vaccines to prevent COVID-19. A number of reports describe the severe public health challenges presented by COVID-19, and the currently available treatment options. See, e.g., Kalorama Information, “COVID-19 Update: Molecular Diagnostics, Immunoassays, Vaccines, Telehealth and Other Areas” (Apr. 7, 2020).

Among such treatments, dissolvable microneedle arrays have be used to deliver recombinant coronavirus vaccines. See, e.g., E. Kim et al., Microneedle array delivered recombinant coronavirus vaccines: Immunogenicity and rapid translational development, EBioMedicine (2020), https://doi.org/10_1016/j.ebiom.2020.102743. However, such dissolvable microneedles have a number of drawbacks, including low mechanical strength and breakage, and the propensity to lose tip sharpness due to the limitations of the molding process. In addition, such dissolvable microneedles are confined to larger thicknesses (e.g., 500 micrometers or higher), which makes it more difficult to conform to patients' skin surfaces. Further, such lab scale fabrication does not translate to large scale manufacturing, which is much more challenging in terms of, inter alia, process and product quality assurance and control.

There is therefore a need in the art for an effective method of vaccine administration through transdermal delivery in which the patch can be accurately and evenly coated, without causing issues of residual vaccine formulation on the array or issues of manufacturing inconsistencies, such as uneven formulation coating on an array or difficulty with formulation sticking to the patch. Many attempts have been made to use transdermal microneedle patches for effective bioactive agent/drug delivery; however, achieving rapid release of bioactive agents from microneedle systems, optimizing and developing effective microneedle shapes and sizes, while also containing a sufficient dosage of bioactive agent has proved elusive. There is thus a need to address issues of viscosity, bioactive agent loading, surface tension, shape and size of microneedles, and common manufacturing defects.

Additionally, there is a need for vaccine products that can be easily self-administered without having to visit a doctor's office or other crowded place that puts patients and healthcare providers at risk of virus exposure. Other needs include avoiding sharp needles typically used for subcutaneous and intramuscular injection and associated biohazard risks, short wear time, and room temperature stable products to avoid the need for cold chain storage.

### SUMMARY

The present disclosure satisfies the above needs, and relates to compositions, devices, methods of treatment, kits and methods of manufacture of pharmaceutical products useful in the treatment of a variety of health conditions, including vaccination against coronavirus and influenza.

More specifically, the disclosure is directed to administration of coronavirus vaccine and/or influenza vaccine as the bioactive agent (active pharmaceutical ingredient) to a subject in need thereof. The present disclosure is directed to transdermally or intracutaneously, or otherwise through the skin, administering a therapeutically effective dose of a coronavirus vaccine and/or influenza vaccine that is easy to use and portable for rapid administration, i.e., by intracutaneous administration via microneedle administration. In one embodiment, the transdermal delivery of coronavirus vaccine and/or influenza vaccine generally comprises a patch assembly having a microprojection member that includes a plurality of microprojections (or “needles” or “microneedles” or “array”) that are coated with, in fluid contact with a reservoir of, or otherwise comprise the vaccine. The patch assembly further comprises an adhesive component, and in a preferred embodiment the microprojection member and adhesive component are mounted in a retainer ring. The microprojections are applied to the skin to deliver the vaccine to the bloodstream, or more particularly, are adapted to penetrate or pierce the stratum corneum at a depth sufficient to provide a therapeutically effective amount to the bloodstream. In one embodiment, the insertion of the vaccine-coated microneedles into the skin is controlled by a handheld applicator that imparts sufficient impact energy density in less than about 10 milliseconds.

Preferably, the microprojection member includes a biocompatible coating formulation comprising the vaccine in a dose sufficient to provide therapeutic effect, e.g., production of coronavirus specific IgG antibodies and other relevant antibodies in the serum of vaccinated mammals as measured by ELISA and virus neutralization assays.

The coating may further comprise one or more excipients or carriers to facilitate the administration of the vaccine across the skin. For instance, the biocompatible coating formulation comprises vaccine and a water-soluble carrier that is first applied to the microprojections in liquid form and then dried to form a solid biocompatible coating. The vaccine patches disclosed herein are easy to self-administer, have short wear times (e.g., 5-30 minutes), are dose-sparing as compared to intramuscularly (IM) or subcutaneously (SC) injected vaccine counterparts, are disposable, and result in at least 50% of patients using the patch being vaccinated/seroconverted. Further, the patches are preservative-free and cause minimal adverse events.

Additional embodiments of the present devices, compositions, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The foregoing features of embodiments will be more readily understood by reference to the following detailed description, taken with reference to the accompanying drawings, in which:

FIG. 1 depicts Applicant Zosano's transdermal microprojection delivery system: (a) applicator; (b) drug-coated patch; (c) microprojection array; and (d) detail of microprojection tip.

FIG. 2 is a line graph showing the solution viscosity of three vaccines formulations: 50 mg/mL HA and sucrose (♦);40 mg/mL HA and sucrose (▪); 35 mg/mL HA and sucrose (▴).

FIG. 3 is a series of micrographs depicting the coating morphology of the flu vaccine coated array as follows: (a) top view of a section of coated array; (b) side view of one microprojection; (c) top view of one microprojection; and (d) front view of one microprojection.

FIG. 4 shows the SDS-PAGE/Western blot analysis of in-process vaccine materials with sheep anti-HA antibody: (a) non-reducing conditions; and (b) reducing conditions.

FIG. 5 is a bar graph of Stability of Systems produced for Phase I Clinical Trial stored for 12 months at 5° C. and 25° C.

### DETAILED DESCRIPTION

Various aspects and embodiments will be described herein. These aspects and embodiments may, however, be embodied in many different forms and should not be construed as limiting; rather, these embodiments are provided so the disclosure will be as thorough and complete so as to inform a person of skill how to make and use the compositions, devices, methods of treatment, kits and methods of manufacture of pharmaceutical products described herein. The terminology used herein is for the purpose of describing the compositions, devices, methods of treatment, kits and methods of manufacture described herein, and is not intended to be limiting unless expressly stated, because the scope of the invention will be limited only by claims accompanying this application and claims accompanying continuation and divisional applications derived from this application. All books, publications, patents, and patent applications cited herein are hereby incorporated by reference in their entirety.

As can be appreciated from the foregoing and following description, each and every feature described herein, and each and every combination of two or more of such features, is included within the scope of the present disclosure provided that the features included in such a combination are not mutually inconsistent. For example, any embodiment whose use is consistent with any other embodiment is contemplated and thus included in this description. Other aspects and embodiments are set forth in the following description and claims, and also when considered in conjunction with the accompanying examples and drawings.

As used in this specification and the appended claims, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure.

### A. DEFINITIONS

Unless defined otherwise, all terms and phrases used herein include the meanings that the terms and phrases have attained in the art, unless the contrary is clearly indicated or clearly apparent from the context in which the term or phrase is used. Any methods and materials similar or equivalent to those described herein can be used in the practice or testing of the present invention, including the particular methods and materials described herein.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon factors known to those skilled in the art. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden or narrow a particular numerical value or range. As a general matter, “about” or “approximately” broaden the numerical value. The disclosure of ranges is intended as a continuous range including every value between the minimum and maximum values plus the broadening of the range afforded by the use of the term “about” or “approximately.” Consequently, recitation of ranges of values herein are intended to serve as a shorthand method of referring individually to each separate value falling within the range, and each separate value is incorporated into the specification as if it were individually recited herein.

The term “biocompatible coating,” as used herein, means and includes a coating formed from a “coating formulation” that has sufficient adhesion characteristics and no (or minimal) adverse interactions with the biologically active agent (a/k/a active pharmaceutical ingredient, or therapeutic agent, or antigen, or drug).

The term “coronavirus” refers to a family of zoonotic viruses that affect humans and cause respiratory tract infections such as common cold symptoms and more severe or even fatal conditions, e.g., severe pneumonia and ARDS. Examples of coronaviruses include alphacoronavirus, betacoronavirus, hCoV-229E, hCoV-NL63, hCoV-OC43, HCoV-HKU1, SARS-COV, MERS-COV, and SARS-COV-2. In some embodiments, the coronavirus is a betacoronavirus having a genome sequence of SARS-COV-2. In other embodiments, the genome sequence of the coronavirus has at least 50%, at least 60%, at least 70%, at least 80%, at least 90%, at least 95%, at least 98% or at least 99% sequence identity with SARS-COV-2. In another aspect, the genome sequence of the coronavirus has at least 50%, at least 60%, at least 70%, at least 80%, at least 90%, at least 95%, at least 98% or at least 99% sequence identity with a bat SARS-like CoV (bat-SL-CoVZC45, MG772933.1). The treatments described herein are useful in vaccinating against all such coronavirus infections and the symptoms arising thereof.

The term “coronavirus vaccine,” as used herein, means any vaccine to coronavirus. For instance, any vaccine that produces coronavirus specific IgG antibodies or other relevant antibodies in the serum of vaccinated mammals as measured by ELISA and virus neutralization assays, including but not limited to, vaccines comprising coronavirus spike (S) protein, SARS-COV-S1 subunit, MERS-S1 subunit, and the vaccines in development listed elsewhere herein.

The term “COVID-19” refers to a respiratory tract infection caused by a newly emergent coronavirus, SARS-COV-2, that was first recognized in Wuhan, China in December 2019. Clinical syndromes of COVID-19 range from mild or uncomplicated illness such as fever, fatigue, cough (with or without sputum production), anorexia, malaise, muscle pain, sore throat, dyspnea, nasal congestion, headache, or rarely, diarrhea, nausea, and vomiting to severe disease that requires hospitalization and oxygen support or the admission to an intensive care unit and may require mechanical ventilation. In severe cases, COVID-19 can be complicated by lung injury, ARDS, sepsis and septic shock, multi-organ failure, including acute kidney injury and cardiac injury. The most common diagnosis in severe COVID-19 patients is severe pneumonia.

The term “excipients” refers to inert substances that are commonly used as a diluent, vehicle, preservative, binder, stabilizing agent, etc. for bioactive agents and includes, but is not limited to, proteins (e.g., serum albumin, etc.), amino acids (e.g., aspartic acid, glutamic acid, lysine, arginine, glycine, histidine, leucine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See also Remington's Pharmaceutical Sciences, 21^{st} Ed., LWW Publisher (2005) for additional pharmaceutical excipients.

The word “intracutaneous” or “transdermal” as used herein, is a generic term that refers to delivery of an active agent (e.g., a therapeutic agent, such as an antigen, a drug, pharmaceutical, peptide, polypeptide or protein) through the skin to the local tissue or systemic circulatory system without substantial cutting or penetration of the skin, such as cutting with a surgical knife or piercing the skin with a hypodermic needle. Intracutaneous agent delivery includes delivery via passive diffusion as well as delivery based upon external energy sources, such as electricity (e.g., iontophoresis) and ultrasound (e.g., phonophoresis).

The term “intracutaneous flux” or “transdermal flux” as used herein, means the rate of intracutaneous or transdermal delivery of an active agent or drug.

The term “microprojection member” or “microneedle array,” and the like as used herein, generally connotes a microprojection grouping comprising a plurality of microprojections, preferably arranged in an array, for penetrating or piercing the stratum corneum. The microprojection member can be formed by etching or punching a plurality of microprojections from a thin sheet of metal or other rigid material, and folding or bending the microprojections out of the plane of the sheet to form a configuration. The microprojection member could alternatively be fabricated with other materials, including plastics or polymers, such as polyetheretherketone (PEEK). The microprojection member can be formed in other known techniques, such as injecting molding or micro-molding, microelectromechanical systems (MEMS), or by forming one or more strips having microprojections along an edge of each of the strip(s), as disclosed in U.S. Pat. Nos. 6,083,196; 6,091,975; 6,050,988; 6,855,131; 8,753,318; 9,387,315; 9,192,749; 7,963,935; 7,556,821; 9,295,714; 8,361,022; 8,633,159; 7,419,481; 7,131,960; 7,798,987; 7,097,631; 9,421,351; 6,953,589; 6,322,808; 6,083,196; 6,855,372; 7,435,299; 7,087,035; 7,184,826; 7,537,795; 8,663,155, and U.S. Pub. Nos. US20080039775; US20150038897; US20160074644; and US20020016562. As will be appreciated by one having ordinary skill in the art, when a microprojection array is employed, the dose of the therapeutic agent that is delivered can also be varied or manipulated by altering the microprojection array size, density, etc.

The term “microprojections” and “microneedles,” as used interchangeably herein, refers to piercing elements that are adapted to penetrate, pierce or cut into and/or through the stratum corneum into the underlying epidermis layer, or epidermis and dermis layers, of the skin of a living animal, particularly a mammal and, more particularly, a human. In one embodiment of the invention, the piercing elements have a projection length less than 1000 microns. In a further embodiment, the piercing elements have a projection length of less than 500 microns, more preferably less than 400 microns. The microprojections further have a width in the range of approximately 25 to 500 microns and a thickness in the range of approximately 10 to 100 microns. The microprojections may be formed in different shapes, such as needles, blades, pins, punches, and combinations thereof.

The terms “patient” and “subject” are used interchangeably herein and refer to a vertebrate, preferably a mammal. Mammals include, but are not limited to, humans.

A bioactive agent “release rate,” as used herein, refers to the quantity of agent released from a dosage form or pharmaceutical composition per unit time, e.g., micrograms of agent released per hour (mcg/hr) or milligrams of agent released per hour (mg/hr). Agent release rates for dosage forms are typically measured as an in vitro rate of dissolution, i.e., a quantity of agent released from the dosage form or pharmaceutical composition per unit time measured under appropriate conditions and in a suitable fluid.

The term “stable,” as used herein, refers to an agent formulation, means the agent formulation is not subject to undue chemical or physical change, including decomposition, breakdown, or inactivation. “Stable” as used herein, refers to a coating also means mechanically stable, i.e., not subject to undue displacement or loss from the surface upon which the coating is deposited.

The term “therapeutically effective” or “therapeutically effective amount,” as used herein, refer to the amount of the biologically active agent needed to stimulate or initiate the desired beneficial result. The amount of the biologically active agent employed in the coatings of the invention will be that amount necessary to deliver an amount of the biologically active agent needed to achieve the desired result. In practice, this will vary widely depending upon the particular biologically active agent being delivered, the site of delivery, and the dissolution and release kinetics for delivery of the biologically active agent into skin tissues.

### B. INTRACUTANEOUS DELIVERY SYSTEM

The apparatus and method for intracutaneously delivering coronavirus vaccine and/or influenza vaccine in accordance with this invention comprises an intracutaneous delivery system having a microneedle member (or system) having a plurality of microneedles (or array thereof) that are adapted to pierce through the stratum corneum into the underlying epidermis layer, or epidermis and dermis layers.

In one embodiment, the intracutaneous delivery system is a transdermal or intracutaneous active agent delivery technology which comprises a disposable patch comprised of a microprojection member centered on an adhesive backing, and an applicator. The microprojection member comprises titanium (or other rigid material, including a plastic or polymeric material like polyetheretherketone (PEEK)) microneedles that are coated with a dry active agent product formulation. The patch is mounted in a retainer ring to form the patch assembly. The patch assembly is removably mounted in a handheld applicator to form the intracutaneous delivery system. The applicator ensures that the patch is applied with a defined application speed and energy to the site of intracutaneous administration. The applicator may be designed for single use or be reusable. Examples of such technology are described in U.S. Pat. Publ. No. US20190070103, owned by Applicant.

More particularly, the patch can comprise an array of about 3 to 6 cm2 of titanium microneedles approximately 200-350 microns long, coated with a hydrophilic formulation of the relevant bioactive agent (e.g., coronavirus vaccine and/or influenza vaccine), and attached to an adhesive backing. The maximum amount of active agent that can be coated on a patch's microneedle array depends on the active agent or moiety of the formulation, the weight of the excipients in the formulation, and the coatable surface area of the microneedle array. For example, patches with about 1 cm², 2 cm², 3 cm², 4 cm², 5 cm², and 6 cm² microneedle arrays may be employed. The patch is applied with a hand-held applicator that presses the microneedles into the skin to a substantially uniform depth in each application, close to the capillary bed, allowing for dissolution and absorption of the active agent coating, yet short of the nerve endings in the skin. The typical patch wear time is about 5 to 45 minutes or less, decreasing the potential for skin irritation. Nominal applicator energies of about 0.20 to 0.60 joules are generally able to achieve a good balance between sensation on impact and array penetration. The actual kinetic energy at the moment of impact may be less than these nominal values due to incomplete extension of the applicator's spring, energy loss from breaking away the patch from its retainer ring, and other losses, which may comprise approximately total 25% of the nominal.

### 1. Array Design

A number of variables play a role in the type of array utilized for a particular active agent. For example, different shapes (e.g., shapes similar to an arrowhead, hook, conical, or the Washington monument) may enable higher active agent loading capacity, while the length of the microprojections may be increased to provide more driving force for penetration. The stratum corneum has a thickness of about 10-40 microns, and microprojections must have an adequate size, thickness, and shape to penetrate and effect active agent delivery through the stratum corneum. The microprojections penetrate the stratum corneum and the substrate interfaces with the surface of the skin.

In some embodiments, it is advantageous to achieve a thicker coating on the microprojections, which will penetrate the stratum corneum, while avoiding applying coating to the substrate or the base (“streets”) of the array, which will not penetrate the stratum corneum. A larger surface area allows for a thicker coating without extending to the base or streets of the array. In certain cases, the coating is applied only to the microprojections. Further, the higher penetration force required for a more bulky projection with coating may be compensated by a longer length and lower density of projections per cm².

Exemplary intracutaneous delivery systems that may be used in the present disclosure include the active agent delivery technologies described in U.S. Pat. Nos. 6,083,196; 6,091,975; 6,050,988; 6,855,131; 8,753,318; 9,387,315; 9,192,749; 7,963,935; 7,556,821; 9,295,714; 8,361,022; 8,633,159; 7,419,481; 7,131,960; 7,798,987; 7,097,631; 9,421,351; 6,953,589; 6,322,808; 6,083,196; 6,855,372; 7,435,299; 7,087,035; 7,184,826; 7,537,795; 8,663,155, and U.S. Pub. Nos. US20080039775; US20150038897; US20160074644; and US20020016562. The disclosed systems and apparatus employ piercing elements of various shapes and sizes to pierce the outermost layer (i.e., the stratum corneum) of the skin, and thus enhance the agent intracutaneous flux. The piercing elements generally extend perpendicularly from a thin, flat substrate member, such as a pad or sheet. The piercing elements are typically small, some having a microprojection length of only about 25 to 400 microns and a microprojection thickness of about 5 to 50 microns. These tiny piercing/cutting elements make correspondingly small microslits/microcuts in the stratum corneum for enhanced transdermal/intracutaneous agent delivery. The active agent to be delivered is associated with one or more of the microprojections, preferably by coating the microprojections with a virus vaccine-based formulation to form a solid, dry coating, or optionally, by the use of a reservoir that communicates with the stratum corneum after the microslits are formed, or by forming the microprojections from solid virus vaccine-based formulations that dissolve after application. The microprojections can be solid or can be hollow, and can further include device features adapted to receive and/or enhance the volume of the coating, such as apertures, grooves, surface irregularities or similar modifications, wherein the features provide increased surface area upon which a greater amount of coating can be deposited. The microneedles may be constructed out of stainless steel, titanium, nickel titanium alloys, or similar biocompatible materials, such as polymeric materials.

The present disclosure therefore encompasses patches and microneedle arrays having the following features:

Patch size: About 1 to 20 cm², or about 2 to 15 cm², or about 4 to 11 cm², or about 3 cm², or about 5 cm², or about 10 cm².

Substrate size: About 0.5 to 10 cm², or about 2 to 8 cm², or about 3 to 6 cm², or about 3 cm², or about 3.13 cm², or about 6 cm².

Array size: About 0.5 to 10 cm², or about 2 to 8 cm², or about 2.5 to 6 cm², or about 2.7 cm², or about 5.5 cm². or about 2.74 cm², or about 5.48 cm².

Density (microprojections/cm²): At least about 10 microprojections/cm², or in the range of about 200 to 2000 microprojections/cm², or about 500 to 1000 microprojections/cm², or about 650 to 800 microprojections/cm², or approximately 725 microprojections/cm².

Number of microprojections/array: About 100 to 4000, or about 1000 to 3000, or about or about 1500 to 2500, or about 1900 to 2100, or about 2000, or about 1987, or about 200 to 8000, or about 3000 to 5000, or about or about 3500 to 4500, or about 4900 to 4100, or about 4000, or about 3974.

Microprojection length: About 25 to 600 microns (micrometers), or about 100 to 500 microns, or about 300 to 450 microns, or about 320 to 410 microns, or about 340 microns, or about 390 microns, or about 387 microns. In other embodiments, the length is less than 1000 microns, or less than 700 microns, or less than 500 microns. Accordingly, the microneedles penetrate the skin to about 25 to 1000 microns.

Tip length: About 100 to 250 microns, or about 130 to about 200 microns, or about 150 to 180 microns, or about 160 to 170 microns, or about 165 microns.

Microprojection width: About 10 to 500 microns, or about 50 to 300 microns, or about 75 to 200 microns, or about 90 to 160 microns, or about 250 to 400 microns, or about 300 microns, or about 100 microns, or about 110 microns, or about 120 microns, or about 130 microns, or about 140 microns, or about 150 microns

Microprojection thickness: about 1 micron to about 500 microns, or about 5 microns to 300 microns, or about 10 microns to 100 microns, or about 10 microns to 50 microns, or about 20 microns to 30 microns, or about 25 microns.

Tip angle: about 10 to 70 degrees, or about 20 to 60 degrees or about 30 to 50 degrees, or about 35 to 45 degrees, or about 40 degrees.

Total active agent per array: About 1 mcg to 500 mcg, or about 10 mcg to 400 mcg, or about 25 mcg to 300 mcg, or at least 50 mcg, or at least 75 mcg, or at least 100 mcg.

Amount of inactive ingredient per array: About 0.1 to 10 mg, or about 0.2 to 4 mg, or about 0.3 mg to 2 mg, or about 0.6 mg, or about 0.63 mg, or about 1.3 mg, or about 1.26 mg. Alternatively, the amount of inactive ingredient is from one to three times less than the active agent, or from about 0.033 mg to about 3.33 mg.

Coating Thickness: about 50 micrometers to about 500 micrometers, or about 100 micrometers to about 350 micrometers, or about 50 micrometers to about 200 micrometers.

Active agent per microprojection: The amount of antigen per microprojection can range from about 13 ng to about 250 ng, or about 0.01 μg to about 100 μg, or about 0.1 to 10 μg, or about 0.5 to 2 μg, or about 1 μg, or about 0.96 μg.

In one embodiment of the invention, the microneedle member has a microneedle density of at least approximately 10 microprojections/cm², more preferably, in the range of at least approximately 200 to 750 microprojections/cm².

In one embodiment of the invention, the microprojections have a projection length less than 1000 microns. In a further embodiment, the microprojections have a projection length of less than 700 microns. In other embodiments, the microprojections have a projection length of less than 500 microns. Preferably, the microprojection length is between 300 and 400 microns in length. The microprojections further have a width in the range of about 100 to about 150 microns and a thickness in the range of about 10 to about 40 microns.

In one embodiment, the microprojection member is constructed out of stainless steel, titanium, nickel titanium alloys, or similar biocompatible materials, such as polymeric materials.

In one embodiment of the invention, the microprojection member includes a biocompatible coating that is disposed on at least the microneedles. The amount of vaccine antigen may be between about 25 to about 500 mcg per array.

Another embodiment has a patch area of about 5 cm² adhered to a titanium substrate with an area of about 3.1 cm² and a thickness of about 25 micrometers. The substrate is comprised of a microprojection array with an area of about 2.74 cm² containing about 1987 microprojections at a density of about 725 microprojections/cm². The dry formulation contained on each microprojection may have the approximate shape of an American football with a thickness that tapers down from a maximum of about 270 μm and comprises about 0.002 μg to about 0.25 μg of coronavirus vaccine antigen per microprojection and about 5 μg to about 500 μg antigen per patch.

Another embodiment has a patch area of about 5 cm² adhered to a titanium substrate of about 6 cm² to and a thickness of about 25 μm. The substrate is comprised of an array with an area of about 5.5 cm² containing about 4000 microprojections at a density of about 725 microprojections/cm². The dry formulation contained on each microprojection is in the approximate shape of an American football with a thickness that tapers down from a maximum of about 270 and consists of about 0.00125 μg to about 0.125 μg of coronavirus vaccine antigen. The microprojections have a length of about 387±13 μm, a width of about 120±13 μm, and a thickness of about 25.4±2.5 μm. The microprojections are rectangular, with a triangular tip to facilitate penetration. The tip has an angle of 40±5 degrees, and is about 165±25 microns long. Further examples of such technology are described in U.S. Pat. Publ. No. US20190070103, owned by Applicant.

The exact combination of bulk, length, and density that produces the desired penetration will vary, and may depend on the active agent, its dose, the disease or condition to be treated and the frequency of administration. Thus, the active agent delivery efficiency of a particular array (i.e., the amount of active agent delivered to the bloodstream) will vary between about 40% to 100%, or about 40%, or about 50%, or about 60%, or about 70%, or about 80%, or about 90%, or about 100%.

### 2. Impact Applicator

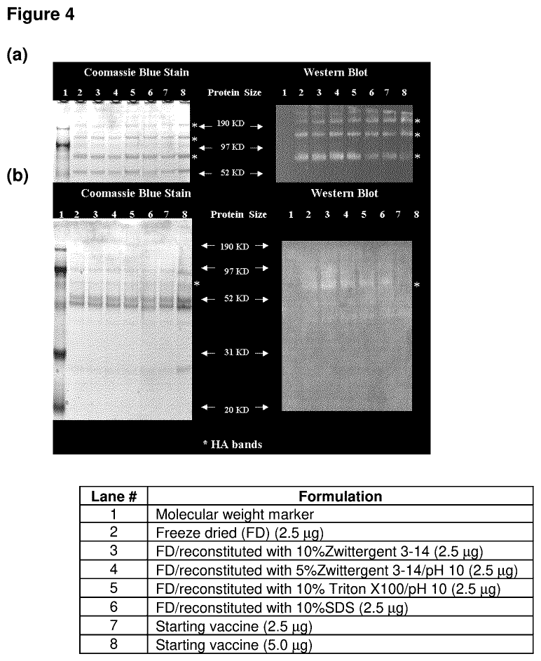

As illustrated in FIGS. 4(A)-(B), 5(A)-(E) of U.S. Pat. Publ. No. US20190070103, owned by Applicant, the intracutaneous active agent delivery system of the present disclosure may further comprise an impact applicator having a body and a piston movable within the body, wherein the surface of the piston impacts the patch against the skin causing the microprojections to pierce the stratum corneum. The applicator is adapted to apply the microneedle array to the stratum corneum with an impact energy density of at least 0.05 joules per cm² in 10 milliseconds or less, or about 0.26 joules per cm² in 10 milliseconds or less, or about 0.52 joules per cm² in 10 milliseconds or less.

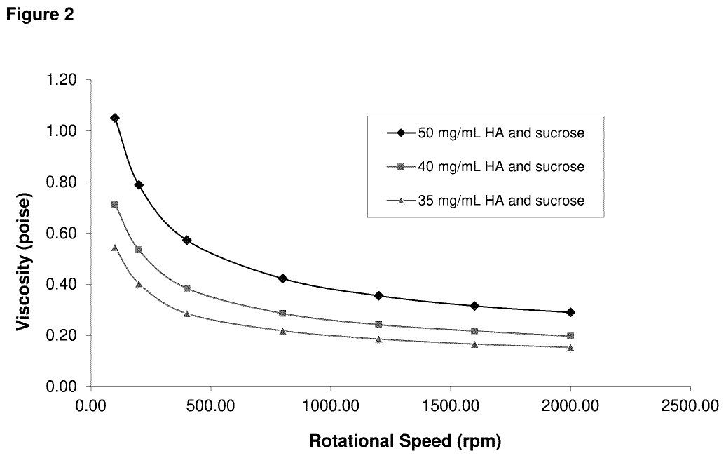

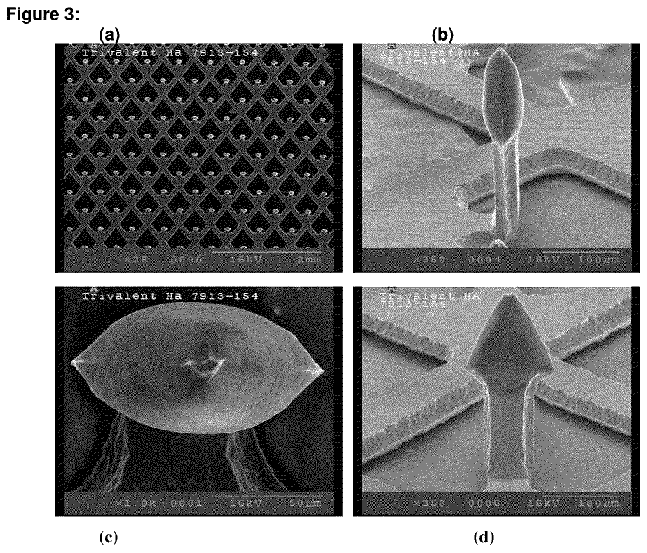

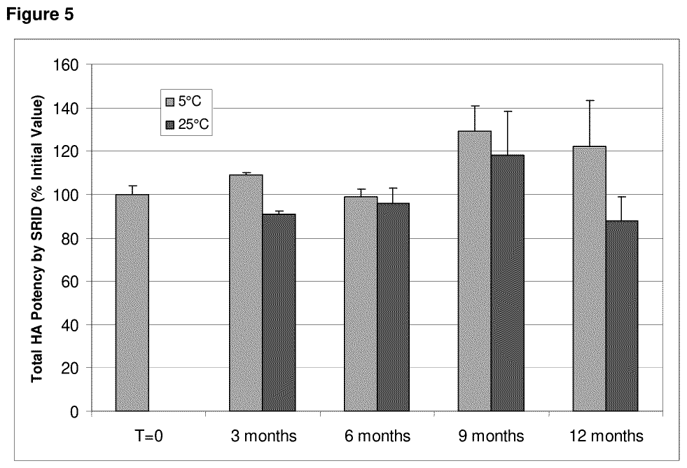

As illustrated in FIGS. 2(A) and 2(B) U.S. Pat. Publ. No. US20190070103, the intracutaneous delivery system comprises a patch having an adhesive backing on one surface and a shiny metal surface on the other side comprised of the array of active agent-coated microneedles. The patch may be applied to the skin by pressing the shiny metal surface against the skin either manually, or preferably by an applicator. Preferably, the applicator applies the patch to the skin with an impact energy density of 0.26 joules per cm² in 10 milliseconds or less. As shown on FIGS. 2A, 2B, 3A and 3B U.S. Pat. Publ. No. US20190070103, the patch may be connected to and supported by a retainer ring structure forming a patch assembly. The retainer ring is adapted to fit onto the impact adaptor and removably attach the patch to the applicator. The retainer ring structure may comprise an inner ring and outer ring, which are designed to receive the adhesive patch and microneedle array. FIGS. 5(A)-(E) of U.S. Pat. Publ. No. US20190070103, demonstrate one embodiment of the claimed invention, in which the user facilitates the connection of the impact applicator to the retainer ring, which is already loaded with the patch and the microneedle array. As shown, once the retainer ring and impact applicator are connected, a user can unlock the impact applicator by twisting the applicator cap. FIG. 5(C) U.S. Pat. Publ. No. US20190070103 shows that the user may then press the applicator downward on the skin to dispense the patch and apply it to the skin. The patch will removably attach to the patient's skin, and the retainer ring remains attached to the applicator. As shown in FIGS. 4(A) and 4(B) U.S. Pat. Publ. No. US20190070103, the retainer ring reversibly attaches to the impact applicator such that the impact applicator can be reused during subsequent dosing events with additional patch assemblies and potentially for other active ingredients and disease states.

In another embodiment, the patch and applicator are supplied as a single, integrated unit, with packaging that ensures the stability and sterility of the formulation. The user removes the system from the packaging and applies the patch as described herein. The used applicator is then disposed of in the regular trash. This embodiment provides a system that is less complex, smaller, lighter, and easier to use.

The present disclosure can also be employed in conjunction with a wide variety of active transdermal systems (as opposed to passive, manual intracutaneous delivery devices described herein), as the disclosure is not limited in any way in this regard.

Some active transdermal systems utilize electrotransport. Illustrative electrotransport active agent delivery systems are disclosed in U.S. Pat. Nos. 5,147,296; 5,080,646; 5,169,382 and 5,169,383. One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane. In many instances, more than one of the noted processes may be occurring simultaneously to different extents. Accordingly, the term “electrotransport” is given herein its broadest reasonable interpretation, to include the electrically induced or enhanced transport of at least one charged or uncharged agent, or mixtures thereof, regardless of the specific mechanism(s) by which the agent is actually being transported with.

In addition, any other transport enhancing method, including but not limited to chemical penetration enhancement, laser ablation, heat, ultrasound, or piezoelectric devices, can be used in conjunction with the disclosure herein.

### 3. Vaccines as Active Agents and Biocompatible Coating

The coating formulations applied to the microprojection member described above to form solid coatings are comprised of a liquid, preferably an aqueous formulation having at least one biologically active agent, which can be dissolved within a biocompatible carrier or suspended within the carrier. The formulation is then coated on the microprojections, dried, sterilized and packaged. The biologically active agent may be influenza vaccine or coronavirus vaccine, such as SARS-Cov-2 subunit vaccine.

The present disclosure encompasses the at least 78 confirmed COVID-19 vaccine candidates, 5 of which have already entered clinical trials. Kalorama paper, supra. Such examples of coronavirus vaccines useful in the present invention include, but are not limited to, the following:
1. Moderna MRNA-127.
2. Inovio Pharmaceuticals INO-4800.
3. Shenzhen Geno-Immune Medical Institute LV-SMENP-DC vaccine.
4. CanSino Biologics Ad5-nCOV.
5. Glaxo and collaborations with Clover Biopharmaceuticals (COVID-19 S-Trimer) and Coalition for Epidemic Preparedness (CEPI) (molecular clamp).
6. Sanofi and collaboration with Translate Bio.
7. Emergent BioSolutions and agreement with Vaxart.
8. Seqiris MF59.
9. Immune Response BioPharma (IRBP) IR101C.
10. Johnson & Johnson.
11. Mitsubishi Tanabe/Medicago.
12. Serum Institute and partnership with Codagenix.
13. Takeda anti-SARS-COV-2 polyclonal hyperimmune globulin.
14. Sengenics.
15. Akers Biosciences.
16. U. of Pittsburgh.
17. Inovio and Ology Biosciences.
18. Dynavax, Clover partnership.
19. U. of Iowa, U. of Georgia.
20. Applied DNA and Takis Biotech.
21. Sanofi and GSK.

A more detailed summary of the vaccines against COVID-19 and part of this disclosure are summarized below.

Select Vaccines in Development for COVID-19 (source: Kalomara, supra)

| Developer | Vaccine | Platform | Phase |
|---|---|---|---|
| Moderna | MRN-1273 | mRNA | 1 |
| Inovio | INO-4800 | DNA | 1 |
| Shenzhen Geno-Immune | LV-SMENP-DC | Modified lentiviral | 1 |
| | Covid-19 aAPC | vector | |
| | | Pathogen-specific aAPC | 1 |
| CanSino | Ad5-nCoV | Recombinant | 2 |
| GSK/Clover | COVID-12-S-Trimer | Protein subunit | PC |
| IntelliStem | IPT-001 | peptide | PC |
| Celularity/Sorrento | CYNK-001 | cell mediated | PC |
| Therapeutics | | | |
| Sanofi/BARDA | | Recombinant | PC |
| Bharat Biotech/FluGen | CoroFlu | self-limiting virus | PC |
| NovaVax | | Recombinant | PC |
| | | nanoparticle | |
| Vaxart/Emergent | | Oral recombinant | PC |
| | | VAAST | |
| Seqiris | MF59 | Adjuvant | PC |
| IRBP | RespiResponse | Cell mediated | PC |
| | IR101C | | |
| Dynavax | CpG 1018 | Adjuvant | PC |
| GSK | AS03 | Adjuvant | PC |
| J&J | | Non-replicating viral | PC |
| | | vector | |
| Medicago | | VLP | PC |
| Serum Inst/Codagenix | | Live attenuated | PC |
| Takeda | TAK-888 | Plasma-derived | PC |
| Altimmune | | Non-replicating viral | PC |
| | | vector | |
| CureVac | | mRNA | PC |
| Generex | | Protein subunit | PC |
| Ibio/Beijing CC Pharming | | Protein subunit/plant | PC |
| ImmunoPrecise Antibodies | | B-Cell select | PC |
| LineaRx/Takis | | DNA | PC |
| Tonix | TNX-1800 | Replicating viral vector | PC |
| Acturus | | Eng RNA | PC |
| Entos | Fusogenix | DNA | PC |
| Heat | | Protein subunit | PC |
| Zydus Cadila | | DNA | PC |
| AnGes | | DNA | PC |
| BioNTech/Pfizer | BNT 162 | mRNA | PC |
| VBI | | pan-coronavirus | PC |
| ISR | ISR-50 | | PC |
| Sk Biopharma | | | PC |
| Sinovac | | DNA | PC |
| Greffex | | non-replicating viral | PC |
| | | vector | |
| Cobra Biologics | | DNA | PC |
| GeoVax/BravoVax | | Non-replicating viral | PC |
| | | vector | |
| Akers/Premas | | D-Crypt | PC |
| Moderna | MRN-1273 | mRNA | 1 |
| Inovio | INO-4800 | DNA | 1 |
| Shenzhen Geno-Immune | LV-SMENP-DC | Modified lentiviral | 1 |
| | Covid-19 aAPC | vector | |
| | | Pathogen-specific aAPC | 1 |
| CanSino | Ad5-nCoV | Recombinant | 2 |
| GSK/Clover | COVID-12-S-Trimer | Protein subunit | PC |
| IntelliStem | IPT-001 | peptide | PC |
| Celularity/Sorrento | CYNK-001 | cell mediated | PC |
| Therapeutics | | | |
| Sanofi/BARDA | | Recombinant | PC |
| Bharat Biotech/FluGen | CoroFlu | self-limiting virus | PC |
| NovaVax | | Recombinant | PC |
| | | nanoparticle | |

This disclosure also relates to new flu vaccines, such as Novavax, Inc.'s NanoFlu™, the company's recombinant quadrivalent seasonal influenza vaccine candidate with its proprietary Matrix-M™ adjuvant, for use in adults aged 65 and older. Kalomara, supra.

Such above vaccines/antigens are compatible with the aqueous coating formulations described herein, and may be loaded onto the microprojection arrays in therapeutically effective amounts according to the methods described herein.

The concentration of biologically active ingredient and excipients in the aqueous coating formulation are carefully controlled to achieve the desired amount of the active ingredient with an acceptable coating thickness, avoid wicking of the coating formulation onto the base of the microneedle array, maintain the uniformity of the coating, and ensure stability. In one embodiment, the active agent is present in the coating formulation at a concentration of between about 1% w/w to about 60% w/w, or between about 15% and 60% w/w, or between about 35% and 45% w/w.

Other coating formulation parameters include:
The vaccine antigen may be stabilized with a disaccharide e.g. sucrose or trehalose at about 0.5, 1 or 2 to 1 ratio by mass of disaccharide to antigen. Other disaccharides that may be used are lactose and maltose in amounts sufficient to stabilize the protein.
Coating thickness ranges from about 50 micrometers to about 100 micrometers.
The viscosity of an aqueous formulation containing antigen or a combination of antigens can range from about 50 to about 300 cP.
Other excipients include tartaric acid, citric acid and histidine.
pH range is 4.4 to 7.4.

The formulation may further comprise an acid at a concentration of between about 0.1% w/w to about 20% w/w. Such acid may be selected from tartaric acid, citric acid, succinic acid, malic acid, maleic acid, ascorbic acid, lactic acid, hydrochloric acid, either individually or in combination. In another embodiment, in the coating formulation, the active agent to acid ratio is about 1:1, about 2:1, about 3:1, about 4:1, or about 5:1. The present disclosure further encompasses a coating formulation comprising about 33% w/w coronavirus vaccine base and about 11% w/w tartaric acid. In some embodiments, the acid is one of tartaric acid, citric acid, succinic acid, malic acid or maleic acid, and is present in an amount of about 0.33% to 10% w/w, or about 8.33% to about 16.67% w/w, or about 13.33% w/w, or about 15% w/w, or about 6.67% w/w. In some embodiments, the coating formulation comprises 45% w/w of the active agent, 15% w/w of the acid, and 40% w/w of water.

The vaccine/antigen may be present in the coating formulation at a concentration comprised between about 1% w/w and about 50% w/w and a weak acid (tartaric acid, citric acid, malic acid, or maleic acid) is present in the coating formulation between about 6.67% w/w and about16.67% w/w.

In certain embodiments, the coating formulations of the present disclosure are free of preservatives.

Surfactants may be included in the coating formulation. Surfactants suitable for inclusion in the coating formulations include, but are not limited to, polysorbate 20 and polysorbate 80. Surfactants are commonly used to improve active agent delivery as penetration enhancers. However, Applicant found that surfactants resulted in undulations in the coating formulation, which is indicative of an uneven film and is highly disadvantageous. Applicant found that the need for surfactants and other penetration enhancers can be avoided through the use of the claimed invention-specifically, through the claimed coronavirus vaccine or influenza vaccine transdermal delivery patches. Furthermore, Applicant surprisingly found that microneedle coating avoided wicking, and the coating sufficiently adhered to the microprojections during the manufacturing process of the microneedle arrays, despite the lack of surfactant.

Antioxidants may be included in the coating formulation. Antioxidants suitable for inclusion in the coating formulations include, but are not limited to, methionine, ascorbic acid, and EDTA.

The coating formulation further comprises a liquid, preferably water, in an amount sufficient (qs ad) to bring the formulation to 100% prior to being dried onto the microneedles. The pH of the liquid coating formulation may be below about pH 8. In other cases, the pH is between about pH 3 and 7.4, or about pH 3.5 to 4.5. Preferably, the pH of the coating formulation is below about pH 8. More preferably, the pH of the coating formulation is comprised between 3 and 7.4. Even more preferably, the pH of the coating formulation is comprised between 3.5 and 5.5.

The liquid coating formulations according to the present disclosure generally exhibit the ability to consistently coat the microneedles with adequate content and morphology, and result in a stable solid-state (dried) formulation, containing less than 5% water, preferably less than 3%. The liquid formulations are applied to the microneedle arrays and the microprojection tips thereof using an engineered coater which allows accurate control of the depth of the microprojection tips dipping into the liquid film. Examples of suitable coating techniques are described in U.S. Pat. No. 6,855,372, included herein by reference in its entirety. Accordingly, the viscosity of the liquid plays a role in microprojection member coating process as has been described. See Ameri, M.; Fan, S C.; Maa, Y F (2010); “Parathyroid hormone PTH(1-34) formulation that enables uniform coating on a novel transdermal microprojection delivery system; ” Pharmaceutical Research, 27, pp. 303-313; see also Ameri M, Wang X, Maa Y F (2010); “Effect of irradiation on parathyroid hormone PTH(1-34) coated on a novel transdermal microprojection delivery system to produce a sterile product adhesive compatibility;” Journal of Pharmaceutical Sciences, 99, 2123-34.

The coating formulations comprising coronavirus vaccine have a viscosity less than approximately 500 centipoise (cP) and greater than 3 cP, or less than approximately 400 cP and greater than 10 cP, or less than approximately 300 cP and greater than 50 cP, or less than 250 cP and greater than approximately 100 cP. In some embodiments, the viscosity of the liquid formulation prior to coating is at least 20 cP. In other embodiments, the viscosity is about 25 cP, or about 30 cP, or about 35 cP, or about 40 cP, or about 45 cP, or about 50 cP, or about 55 cP, or about 60 cP, or about 65 cP, or about 70 cP, or about 75 cP, or about 80 cP, or about 85 cP, or about 90 cP, or about 95 cP, or about 100 cP, or about 150 cP, or about 200 cP, or about 300 cP, or about 400 cP, or about 500 cP. In other embodiments, the viscosity is more than about 25 cP, or a more than about 30 cP, or more than about 35 cP, or more than about 40 cP, or more than about 45 cP, or more than about 50 cP, or more than about 55 cP, or more than about 60 cP, or more than about 65 cP, or more than about 70 cP, or more than about 75 cP, or more than about 80 cP, or more than about 85 cP, or more than about 90 cP, or more than about 95 cP, or more than about 100 cP, or more than about 150 cP, or more than about 200 cP, or more than about 300 cP, or more than about 400 cP, or less than about 500 cP. In a preferred embodiment, the viscosity of the coating formulation is more than about 80 cP and less than about 350 cP; in another preferred embodiment, the viscosity is more than about 100 cP and less than about 350 cP; and, in another preferred embodiment, the viscosity is more than about 100 cP and less than about 250 cP.

Once applied to the microprojections, the coating formulation may have an average thickness of about 10 to about 400 microns, or from about 30 to about 300 microns, or from about 100 microns to about 175 microns, or from about 115 to about 150 microns, or about 135 microns, as measured from the microprojection surface. Although it is preferable that the coating formulation have a uniform thickness covering the microprojection, the formulation may vary slightly as a result of the manufacturing process. The microprojections are generally coated uniformly because they penetrate the stratum corneum. In some embodiments, the microprojections are not coated the entire distance from the tip to the base; instead, the coating covers a portion of the length of the microprojection, measured from tip to the base, of at least about 10% to about 80%, or 20% to about 70%, or about 30% to about 60%, or about 40% to about 50% of the length of the microprojection.

The liquid coating formulation is applied to an array of microprojections so as to deliver a dose of the active agent in the amount of about 1 mcg to about 500 mcg per array. In the case of coronavirus vaccine, the dose is about 5 mcg to about 500 mcg, or about 25 mcg to about 500 mcg delivered to the stratum corneum per array (via a patch or other form). The microprojection shape and size has a significant bearing on the active agent loading capacity and on the effectiveness of active agent delivery.

In one aspect, the aqueous vaccine formulations are pre-formulated by (a) diafiltration/concentration; (b) lyophilization; and (c) reconstitution.

After reconstitution, the aqueous vaccine formulations are dried onto the microprojections into a solid coating, generally by drying a coating formulation on the microprojection, as described in U.S. Application Pub. No. 2002/0128599. The coating formulation is usually an aqueous formulation. During a drying process, all volatiles, including water are mostly removed; however, the final solid coating may still contain about 1% w/w water, or about 2% w/w water, or about 3% w/w water, or about 4% w/w water, or about 5% w/w water. The oxygen and/or water content present in the formulations are reduced by the use of a dry inert atmosphere and/or a partial vacuum. In a solid coating on a microprojection array, the active agent antigen may be present in an amount of less than about 500 mcg per unit dose (patch) or less than about 400 mcg or less than about 300 mcg or less than about 200 mcg or less than about 100 mcg. With the addition of excipients, the total mass of solid coating may be less than about 5 mg per unit dose, or less than about 2 mg per unit dose.

The microprotrusion member is usually present on an adhesive backing, which is attached to a disposable polymeric retainer ring. This assembly is packaged individually in a pouch or a polymeric housing. In addition to the assembly, this package contains a dead volume that represents a volume of at least 3 mL. This large volume (as compared to that of the coating) acts as a partial sink for water. For example, at 20° C., the amount of water present in a 3 mL atmosphere as a result of its vapor pressure would be about 0.05 mg at saturation, which is typically the amount of residual water that is present in the solid coating after drying. Therefore, storage in a dry inert atmosphere and/or a partial vacuum will further reduce the water content of the coating resulting in improved stability.

According to the disclosure, the coating can be applied to the microprojections by a variety of known methods. For example, the coating may be only applied to those portions of the microprojection member or microprojections that pierce the skin (e.g., tips). The coating is then dried to form a solid coating. One such coating method comprises dip-coating. Dip-coating can be described as a method to coat the microprojections by partially or totally immersing the microprojections into a coating solution. By use of a partial immersion technique, it is possible to limit the coating to only the tips of the microprojections.

A further coating method comprises roller coating, which employs a roller coating mechanism that similarly limits the coating to the tips of the microprojections. The roller coating method is disclosed in U.S. Application Pub. No. 2002/0132054. As discussed in detail therein, the disclosed roller coating method provides a smooth coating that is not easily dislodged from the microprojections during skin piercing.

A further coating method that can be employed within the scope of the present invention comprises spray coating. Spray coating can encompass formation of an aerosol suspension of the coating composition. In one embodiment, an aerosol suspension having a droplet size of about 10 to 200 picoliters is sprayed onto the microprojections and then dried.

Pattern coating can also be employed to coat the microprojections. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In one embodiment of the disclosure, the thickness of the dried coating formulations comprising coronavirus vaccine or influenza vaccine range from about 10 to 100 microns as measured from the microprojection surface, or from about 20 to 80 microns, or from about 30 to 60 microns, or from about 40 to 50 microns. The desired coating thickness is dependent upon several factors, including the required dose and, hence, coating thickness necessary to deliver the dose, the density of the microprojections per unit area of the sheet, the viscosity, the solubility and concentration of the coating composition and the coating method chosen. The thickness of coating applied to microprojections can also be adapted to optimize stability of the coronavirus vaccine. Known formulation adjuvants can also be added to the coating formulations provided they do not adversely affect the necessary solubility and viscosity characteristics of the coating formulation nor the physical integrity of the dried coating.

The coating is applied to the microneedles, which protrude from the base, or streets, of the microneedle array. The coating is applied to the tips of the microneedles, and is not intended to cover the microneedles and the surface of the microneedle array. This reduces the amount of active agent per transdermal patch, which is advantageous in light of FDA Guidance on the danger of residual active agent on transdermal delivery systems, which suggests that the amount of residual active agent in a system should be minimized. See FDA Guidance for Industry, Residual Drug in Transdermal and Related Drug Delivery Systems (August 2011). Applicant's strategy was to maximize active agent release into skin per unit area, without using an excess of active agent for coating.

After a coating has been applied, the coating formulation is dried onto the microprojections by various means. The coated microprojection member may be dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

Coating was conducted at ambient temperature utilizing a roller drum, rotating at 50 rpm, in an active agent formulation reservoir (2 mL in volume) to produce a film of controlled thickness of around 50 to 100 μm in thickness. Further information about the coating process can be found in U.S. Pat. No. 6,855,372. Microprojection arrays are dipped into the active agent film, and the amount of coating is controlled by the number of dips (passes) through the active agent film.

During the drying process, there may be issues related to forming a uniform coating the microprojection with a controlled and consistent thickness. One common issue in transdermal patch coating, called “dripping” or “teardrop” formations, occurs when the coating is drying and the coating accumulates at the end of the microprojections in a “teardrop” shape. This teardrop shape can blunt the sharp end of the microneedle, potentially impacting the effectiveness and uniformity of penetration. Uneven layers of formulation on the microprojections results in uneven, and sometimes inadequate active agent delivery. Additionally, the issues in the drying process cause issues of quality control in formulation coating.

Liquid coating formulations comprise coronavirus vaccine/antigen or influenza vaccine/antigen in an amount of about 10 to about 1000 mcg HA/mL, or about 25 to about 500 mcg HA/mL, or an amount of 0.001% w/w to about 30% w/w, or about 0.01% w/w to about 25% w/w, or about 0.1% w/w to about 10% w/w, and tartaric acid in an amount of about 5% w/w to about 25% w/w, preferably about 10% w/w to about 20% w/w, more preferably about 15% w/w, in a liquid carrier, preferably water, more preferably deionized water. With these liquid coating formulations, maintaining a viscosity of about 150 cP to about 350 cP, preferably about 200 cP to about 300 cP, more preferably about 250 centipoise, and a surface tension of about 50 mNm⁻¹ to about 72 mNm⁻¹, preferably about 55 mNm⁻¹ to about 65 mNm⁻¹, more preferably about 62.5 mNm⁻¹ is resistant to dripping. Teardrop formation can be avoided while simultaneously allowing each dip of microprojections into the liquid coating formulation to pick up sufficient volume of liquid coating formulation, thereby achieving the desired active agent dose with a minimum number of dips. When the viscosity and surface tension of the coating solution are high enough, the coated liquid does not quickly drip back or form a teardrop shape after dipping and before drying.

### 4. Packaging and Sterilization

Improved physical stability of the dry coated formulations provides not only the benefit of an increased storage or shelf life for the therapeutic agent itself, but enhances efficacy in that once stabilized in accordance with the compositions of and methods for formulating and delivering of the present invention, the therapeutic agents become useful in a greater range of possible formulations, and with a greater variety of therapeutic agent delivery means.

The present disclosure comprises an active agent formulation wherein the deterioration by oxygen and/or water is minimized and/or controlled by the manufacture and/or packaging of the active agent formulation in a dry inert atmosphere. The formulation may be contained in a dry inert atmosphere in the presence of a desiccant, optionally in a chamber or package comprising a foil layer.

The desiccant can be any known to those skilled in the art. Some common desiccants include, but are not limited to molecular sieve, calcium oxide, clay desiccant, calcium sulfate, and silica gel. The desiccant may be one that can be placed with the biologically active agent-containing formulation in the presence of an inert atmosphere in a package comprising a foil layer.

In another aspect, the active agent formulation is packaged in a chamber comprising a foil layer after the formulation is coated onto the microprojection array delivery device. In this embodiment, a desiccant is contained in the chamber, preferably attached to a chamber lid which comprises a foil layer, and the chamber is purged with dry nitrogen or argon or other inert gas such as a noble gas prior to the delivery device-containing foil chamber being sealed by the foil lid. Any suitable inert gas can be used herein to create the dry inert atmosphere.

In one embodiment, the compositions of and methods for formulating and delivering coronavirus vaccine suitable for intracutaneous delivery utilize a patch assembly. This patch assembly is manufactured and/or packaged in a dry inert atmosphere, and in the presence of a desiccant. In one embodiment, the patch assembly is manufactured in a dry inert atmosphere and/or packaged in a chamber comprising a foil layer and having a dry inert atmosphere and a desiccant. In one embodiment, the patch assembly is manufactured and/or packaged in a partial vacuum. In one embodiment, the patch assembly is manufactured and/or packaged in a dry inert atmosphere, and a partial vacuum. In one embodiment, patch assembly is manufactured in a dry inert atmosphere under a partial vacuum and/or packaged in a chamber comprising a foil layer and having a dry inert atmosphere, a partial vacuum, and a desiccant.

Generally, in the noted embodiments of the present invention, the inert atmosphere should have essentially zero water content. For example, nitrogen gas of essentially zero water content (dry nitrogen gas) can be prepared by electrically controlled boiling of liquid nitrogen. Purge systems can be also used to reduce moisture or oxygen content. A range for a partial vacuum is from about 0.01 to about 0.3 atmospheres.

In one embodiment, the compositions of and methods for formulating and delivering Coronavirus vaccine suitable for Intracutaneous delivery using a microneedle delivery device, is manufactured and/or packaged in a dry inert atmosphere, preferably nitrogen or argon, and in the presence of a desiccant or oxygen absorber.

In one embodiment, the compositions of and methods for formulating and delivering vaccine suitable for intracutaneous delivery using a microneedle delivery device is manufactured and/or packaged in a foil lined chamber having a dry inert atmosphere, preferably nitrogen, and a desiccant or oxygen absorber.

In one embodiment, the compositions of and methods for formulating and delivering vaccine suitable for intracutaneous delivery using a microneedle delivery device is manufactured and/or packaged in a partial vacuum.

In one embodiment, the compositions of and methods for formulating and delivering vaccine suitable for intracutaneous delivery using a microneedle delivery device is manufactured and/or packaged in a foil lined chamber having a dry inert atmosphere, preferably nitrogen, a partial vacuum, and a desiccant or oxygen absorber.

In an aspect of this embodiment, the vaccine further comprises a biocompatible carrier. In another embodiment, there is an intracutaneous delivery system, adapted to deliver vaccine, comprising: (a) a microprojection member including a plurality of microprojections that are adapted to pierce the stratum corneum of a patient; (b) a hydrogel formulation comprised of coronavirus vaccine, wherein the hydrogel formulation is in communication with the microprojection member; and (c) packaging purged with an inert gas and adapted to control environmental conditions sealed around the microprojection member, wherein the sealed package has been exposed to radiation to sterilize the microprojection member.

In another embodiment, there is an intracutaneous delivery system, adapted to deliver vaccine, comprising: (a) a microprojection member including a plurality of microprojections that are adapted to pierce the stratum corneum of a patient; (b) a solid film disposed proximate the microprojection member, wherein the solid film is made by casting a liquid formulation comprising vaccine, a polymeric material, a plasticizing agent, a surfactant and a volatile solvent; and (c) packaging purged with an inert gas and adapted to control environmental conditions sealed around the microprojection member, wherein the sealed package has been exposed to radiation to sterilize the microprojection member.

The present disclosure is also to a method for terminally sterilizing a patch assembly adapted to deliver vaccine, comprising the steps of: (a) providing a microprojection member having a plurality of microprojections that are adapted to pierce the stratum corneum of a patient having a biocompatible coating comprising coronavirus vaccine disposed on the microprojection member; and (b) exposing the microprojection member to radiation selected from the group consisting of gamma radiation and e-beam, wherein the radiation is sufficient to reach a desired sterility assurance level. Such sterility assurance level may be 10⁻⁶ or 10⁻⁵. The method may further comprise sealing the micro-projection member with a desiccant inside packaging purged with an inert gas and exposing the packaged microprojection member to radiation selected from the group consisting of gamma radiation and e-beam radiation, wherein the radiation is sufficient to reach a desired sterility assurance level.

In an aspect of this embodiment, the method further comprises the step of mounting a patch comprised of a microprojection member attached to an adhesive backing on a pre-dried retainer ring to form a patch assembly, and subsequently sealing the microprojection member inside the packaging. In an aspect of this embodiment, the system further comprises a desiccant sealed inside the packaging with the patch assembly, and/or the packaging is purged with nitrogen, and/or the packaging comprises a pouch comprised of a foil layer. Preferably, the foil layer comprises aluminum.

The step of exposing the microprojection member to radiation may occur at approximately -78.5 to 25° C., or the member may be exposed to radiation at ambient temperature. The radiation may be in the range of approximately 5 to 50 kGy, or approximately 10 to 30 kGy, or approximately 15 to 25 kGy, or approximately 21 kGy, or approximately 7 kGy. In one aspect of this embodiment, the radiation is delivered to the microprojection member at a rate of at least approximately 3.0 kGy/hr.

In one embodiment, vaccine coated microneedles are exposed to a dose of radiation in the range of approximately 7-30 kGy. More preferably in the range of 15-30 kGys to a sterility assurance level of 10-5 to 10-6.

The present disclosure relates to vaccine formulations which, when coated on the microneedle members of the present disclosure, is stable at room temperature for at least 6 months, or at least 9 months, or at least 12 months, or at least 18 months, or at least 24 months after being exposed to radiation as described above.

In certain embodiments, the dried vaccine formulation on the microneedles retains for at least 6 months approximately 100% of initial purity, or approximately 99% of initial purity, or approximately 98% of initial purity, or approximately 97% of initial purity, or approximately 96% of initial purity, or approximately 95% of initial purity, or approximately 90% of initial purity. In other aspects, such purity is retained for at least 9 months, or at least 12 months, or at least 18 months, or at least 24 months after packaging.

In one embodiment, a method for manufacturing a patch assembly for an intracutaneous delivery device adapted to deliver a vaccine, comprises the steps of: providing a microneedle member having a plurality of microneedles that are adapted to penetrate or pierce the stratum corneum of a patient having a biocompatible coating disposed on the microneedle member, the coating being formed from a coating formulation having vaccine, disaccharide, and tartaric acid, citric acid, malic acid or maleic acid disposed thereon; sealing the microneedle member with a desiccant inside packaging purged with nitrogen and adapted to control environmental conditions surrounding the microneedle and exposing the microneedle member to radiation selected from the group consisting of gamma radiation, e-beam and x-ray wherein the radiation is sufficient to reach a desired sterility assurance level.

In accordance with another embodiment of the invention, a method for delivering stable biologically active agent formulations comprises the following steps: (i) providing a microprojection member having a plurality of microprojections, (ii) providing a stabilized formulation of biologically active agent; (iii) forming a biocompatible coating formulation that includes the formulation of stabilized biologically active agent, (iv) coating the microprojection member with the biocompatible coating formulation to form a biocompatible coating; (v) stabilizing the biocompatible coating by drying; and (vi) applying the coated microprojection member to the skin of a subject.

Additionally, optimal stability and shelf life of the agent is attained by a biocompatible coating that is solid and substantially dry. However, the kinetics of the coating dissolution and agent release can vary appreciably depending upon a number of factors. It will be appreciated that in addition to being storage stable, the biocompatible coating should permit desired release of the therapeutic agent.

Encompassed herein is a method for terminally sterilizing a transdermal device adapted to deliver coronavirus vaccine, comprising the steps of: providing a microprojection member having a plurality of microprojections that are adapted to penetrate or pierce the stratum corneum of a patient having a biocompatible coating disposed on the microprojection member, the coating being formed from a coating formulation having vaccine disposed thereon; and exposing the microprojection member to radiation selected from the group consisting of gamma radiation and e-beam, wherein the radiation is sufficient to reach a desired sterility assurance level. A further aspect of this method comprises the further step of sealing the microprojection member inside packaging adapted to control environmental conditions surrounding the microprojection member. In one aspect the packaging comprises a foil pouch. A further aspect of this method, comprises the further step of sealing a desiccant inside the packaging. Further, the method comprises the step of mounting the microprojection member on a pre-dried retainer ring prior to sealing the microprojection member inside the packaging. A further aspect of this method comprises the step of purging the packaging with an inert gas prior to sealing the packaging. In one embodiment, the inert gas comprises nitrogen.

### B. METHODS OF TREATMENT

The active agent-device combinations of the present invention can be used to treat a variety of diseases and conditions, including vaccination against COVID-19, other coronaviruses and influenza. The patient may self-administer the vaccine-coated microarray patch comprising about 5 mcg to about 500 mcg of vaccine/antigen by using the applicator device described elsewhere herein. The patch is applied to a selected area of skin generally flat and free of excess hair, such as the upper arm, near the wrist, thigh, chest or back. The patch wear time may vary from about 1 minute to about 30 minutes, or about 5 minutes to about 20 minutes, or about 10 minutes. Thereafter, the patient removes the patch and discards it into the trash.

The patient may receive the patch from the doctor's office, a pharmacy, through the mail or from an employer. The patch does not require refrigeration, is for single-use and is disposable without the need for sharps biocontainers, etc.

In one embodiment, when the vaccine patch of the present disclosure is administered to a population of patients, a statistically significant number of such patients are successfully vaccinated. In other embodiments, at least 10% of such patients are seroprotected, or at least 20%, or at least 30%, or at least 40%, or at least 50%, or at least 60%, or at least 70%, or at least 80%, or at least 90% of such patients are seroprotected.

In other aspects, the vaccine coated patches described herein are dose-sparing as compared with IM or SC injectable vaccine counterparts. For instance, the patches herein require at least 5%, or at least 10%, or at least 20%, or at least 30% less vaccine/antigen than their IM or SC injectable counterparts.

### EXAMPLES

### Example 1—Formulation Approach that Enables the Coating of a Stable Trivalent Influenza Vaccine on a Transdermal Microprojection Patch

As described below, a trivalent influenza vaccine transdermal patch was successfully developed with three key advantages over trivalent influenza vaccine intramuscular (IM) injection formulation: (1) preservative-free; (2) room-temperature storage; and (3) dose sparing. More importantly, this patch system proved to be stable and efficacious in pre-clinical and clinical studies.

The trivalent influenza vaccine, featuring two type A and one Type B strains of influenza virus with 15 mcg haemagglutinin (HA) as the surface antigen for each strain, is currently marketed in the U.S. in two formulations, an inactivated, injectable form (Fluzone®, Sanofi Pasteur; Fluvirin®, Novartis Vaccine; Fluvarix® and FluLaval™ GlaxoSmithKline; Afluria®, CSL) and a live attenuated nasal spray (FluMist®, Medimmune). The trivalent injectable form is available as a sterile suspension prepared from three individual monovalent strains of influenza virus and is administered by conventional needle and syringe, which may cause undesirable pain and increasing costs due to intransient safety problems associated with sharps. In addition, the liquid injectable products have to be stored under refrigerated conditions requiring costly cold-chain storage throughout the manufacturing process. For the purpose of sterility, the injectable formulation may contain mercury-based thimerosal as a preservative in multi-dose vials. Although FluMist® nasal spray offers an alternative to needle/syringe injection, it still features a liquid formulation requiring cold-chain storage at 2-8° C. Overall, there are strong needs to seek needle-free influenza vaccine immunization alternatives capable of providing additional cost benefit in cold-chain free storage and added safety in a preservative-free dosage form.

Skin contains abundant antigen presenting cells (APCs), the Langerhans cells (LCs) in the viable epidermis, and the dendritic cells in the dermis. APCs play a critical role in picking-up antigens in the skin, migrating into draining lymph nodes, and presenting processed antigens to the CD8+ and CD4+ T helper cells. Therefore, as can now be appreciated by the present disclosure, vaccination via the skin route, i.e., transdermal immunization, makes dose sparing possible, which adds further benefits to patient safety and cost saving. The effectiveness of the skin immune system is responsible for the success and safety of vaccination strategies that have been targeted to the skin by intradermal vaccination of live-attenuated smallpox vaccine and rabies vaccine using one-fifth to one-tenth of the standard intramuscular doses.

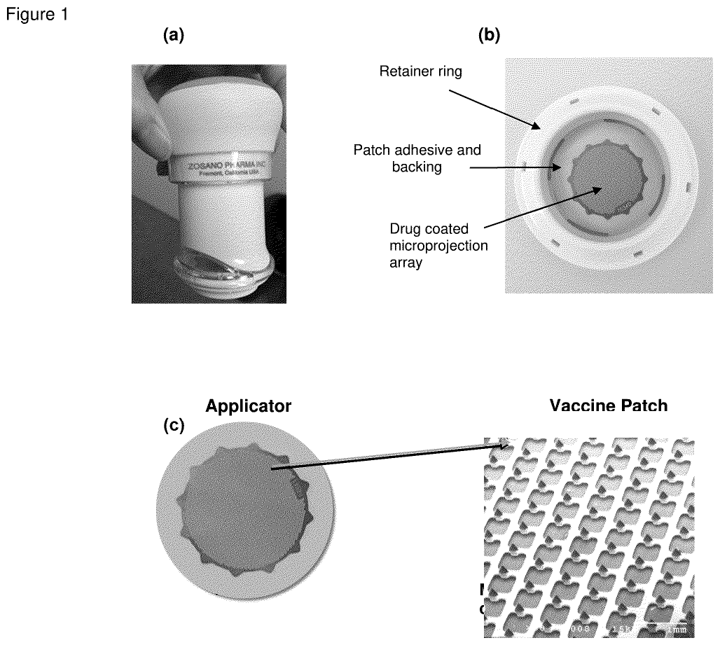

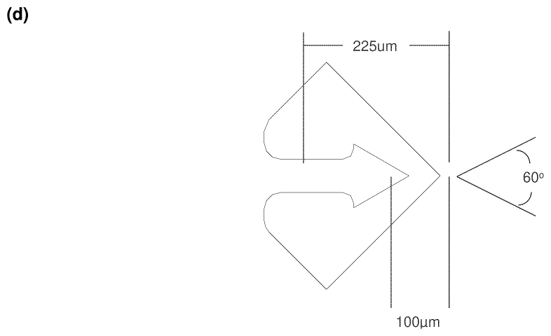

All needs above led to the development of a novel transdermal microprojection patch delivery system for trivalent influenza vaccine. This transdermal microprojection delivery system is capable of penetrating the superficial skin barrier without pain or inconvenience. The small drug-coated patch is 5 cm² in area and seated in a patch retainer ring. The patch is applied with a hand-held reusable applicator (FIG. 1a). The patch comprises a titanium microprojection array (˜1,300 microprojections per 2 cm² in FIG. 1b) attached to the center of an adhesive backing. Vaccine formulation is coated on the tip of each microprojection. The patch and retainer ring is pressed onto the skin. The drug-coated microprojections penetrate through the superficial skin barrier layer into the epidermal/dermal layers (50-150 micrometers in depth), where the vaccine formulation rapidly dissolves and releases into the skin.

The vaccine bulk, i.e., the current liquid injectable product, was reformulated and placed on the microprojection array using a novel coating process which requires high vaccine concentrations and other physical properties (described below). The monovalent strains of influenza virus are low concentration liquids with complex formulations as the result of a complicated vaccine manufacturing process. Each strain of influenza virus is propagated in the allantoic fluid of embryonated chicken eggs. From the allantoic fluid, influenza virus particles are concentrated, purified, disrupted by a detergent (Triton X-100), and then inactivated by the addition of formaldehyde and/or sodium deoxycholate to produce a “split virus” or “split virion” for each of the three strains. The inactivated strains are suspended and combined into the trivalent solution which must be stored under refrigerated conditions throughout the manufacturing and shipping process. Thimerosal or 2-phenoxyethanol (2-PE) is normally added as a preservative in multi-dose vials. Thus, the vaccine bulk may contain insoluble particles (water-insoluble lipids, lipid-protein complexes, and aggregated proteins), Triton X-100, low molecular-weight compounds and buffers.

This Example 1 describes the pre-formulation and formulation process capable of increasing the vaccine concentration by 200-500 fold and defining critical coating parameters to manufacture the patch delivery systems. The patches coated with preservative-free trivalent influenza vaccine were evaluated for long-term stability and tested pre-clinically and in Phase I human clinical trials to demonstrate the feasibility for cold-chain free, room-temperature storage and dose-sparing immunogenicity performance over the intramuscular administration route.

### MATERIALS AND METHODS

### Materials

Monovalent split virion Influenza virus strain extracts were derived from egg incubation. Each monovalent strain solution was further processed prior to use as described below in the Methods section. Sucrose (Lot Number 27412A, High Purity Low Endotoxin Grade) and trehalose (Lot Number 26554A, High Purity Low Endotoxin Grade) were purchased from Ferro-Pfanstiehl (Cleveland, OH) and were used as received. Surfactants were purchased from several suppliers and used as received—Tween 80, Lot Number 58217, (ICN Biomedicals Inc., Aurora, OH); Zwittergent 3-14, Lot Number B36399 (Calbiochem, San Diego, CA); Triton X100, Lot Number QC2755S4D1 (89521), (Union Carbide Corporation, Houston, TX); Pluronic F68, Lot Number 16H1147, (Sigma, St. Louis, MO).

The patch delivery system consists of a 2 cm² titanium array of microprojections (Kemac, Azusa, CA) with 1,300 total microprojections where the length of microprojection is 225 micrometers, the length and the width of the microprojection head is 100 micrometers and 115 micrometers, respectively, and with a tip angle of 60 degrees (see FIG. 1d). The delivery system also consists of a polycarbonate ring (Jatco, Union City, CA), a 5cm² adhesive patch (Medical Tape 1523, 3M, St. Paul, MN), and an aluminum foil pouch (Mangar, New Britain, PA).

### Methods

### Rheometry

Viscosity of the concentrated coating formulations was determined using a cone and plate viscometer (Brookfield Eng. Lab., CAP 2000). Each measurement required 70 μL of a liquid sample. Viscosity was determined at several shear rates and several temperatures for each liquid sample.

### Contact Angle Measurements

The contact angle between the coating formulations and titanium substrates was determined using a contact angle meter (Tantec Inc., Schaumberg, IL) based on a half-angle measuring method by placing a liquid droplet of 5 μL on a metallic titanium sheet.

### Scanning Electron Microscopy (SEM)

SEM was used to determine the morphology and placement of the coating on the microprojections. The coated titanium arrays were adhered to aluminum studs with carbon double-stick tape and placed in the vacuum chamber of the SEM (Hitachi, S-2460N).

### Single Radial Immunodiffusion (SRID)

A single radial immunodiffusion assay was adapted for the quantification of influenza HA content in the starting materials, coating solution and coated arrays. In this passive diffusion method, after treatment with a detergent, the sample solution and the reference vaccine diffuse radially from the wells and react with a specific antibody, which is uniformly dispersed in the gel matrix. The antigen-antibody interaction is manifested by a defined ring of precipitation around the antigen (HA) well. The ring diameter will continue to increase until equilibrium is reached. Under equilibrium conditions, the precipitin ring diameter is proportional to the concentration of HA. After complete diffusion, the circles of precipitations for each sample solution and reference vaccine are measured. The haemagglutinin content in the sample is determined against an international reference provided by Influenza Reference Center and calibrated in μg/mL.

### Enzyme-Linked Immunosorbent Assay (ELISA)

An indirect ELISA method was developed to detect the presence of anti-influenza specific antibodies from sera of hairless guinea pigs (HGPs). Previously, an indirect ELISA was developed to determine the anti-ovalbumin antibody titers in HGPs immunized with ovalbumin coated arrays. For influenza vaccine coated arrays, a similar assay was developed to specifically determine the endpoint titer of sera from HGPs immunized with influenza vaccine. The endpoint titer is defined as the inverse dilution, determined by nonlinear regression, of an immunized HGP serum sample with an OD that is three standard deviations above the mean OD of non-immunized control HGP sera (n=10).

### Bicinchoninic Acid Assay (BCA)

Protein content of raw materials, coating solution and coated arrays was measured by the BCA assay using a kit purchased from Pierce (Rockford, Ill). A set of serially diluted standards was prepared directly from the vaccine raw material. Unknown samples were diluted with water to a concentration that was within the standard working range of the assay. Standards and samples were loaded onto a 96-well plate and placed into a plate reader (Molecular Devices, SpectraMax 250), shaken for 30 seconds, and incubated at 37° C for 30 minutes. The absorbance was measured at 562 nm and the mean values of the standards were fit to a 4-parameter equation of the following form:

$$y = \frac{( A - D )}{{( 1 + x / C )}^{B}} + D$$

### Lowry Assay

The total protein content of some samples was measured by a modified Lowry Assay using bovine serum albumin (BSA) as a protein standard. The Lowry method is based on the formation of a blue complex formed as a result of reaction of protein with copper ions, and the subsequent reduction of the Folin-Ciocalteau reagent by the protein-copper complex. The intensity of the blue color is proportional to the amount of protein present in the sample and is measured spectrophotometrically at 750 nm.

### SDS-PAGE/Western Blot

Flu vaccine HA protein samples were separated by SDS-PAGE on an Invitrogen pre-cast NuPAGE gel. The resolved proteins were blotted onto PVDF membranes according to the Instructions for using the XCell II Blot Module “Novex Western Transfer Apparatus” (Invitrogen). The blotted PVDF membranes were probed with diluted anti-HA primary antibody or anti-HA antiserum. Nonspecific binding sites were blocked by PBS with 5% milk plus 0.1% Tween 20. The Western blot was visualized by using HRP-conjugated secondary antibody and ECL detection reagent from Amersham Pharmacia.

### Assay for Triton-X 100

The concentration of the surfactant Triton-X 100 was measured in liquid samples by two methods, a colorimetric assay and an HPLC method. The colorimetric assay involved forming a complex with ammonium cobaltothiocyanate which formed a blue precipitate. The precipitate was then extracted into ethylene dichloride and the absorbance was measured spectrophotometrically. The HPLC method was a reversed-phase method using a C4 column and linear acetonitrile gradient.

### Tangential-Flow Filtration (TFF)

Two types of the TFF system were used for diafiltration and concentration of the split virion influenza extract: a lab-scale TFF system (Millipore, Labscale) equipped with a Pellicon XL, regenerated cellulose membrane (Millipore, 50 cm2, 30 kD MWCO) and a larger scale TFF system (Pall, Centremate™) equipped with a 0.1 ft2 30 kD MWCO polyethersulfone PES membrane. Tangential flow filtration was employed as a first step to remove the salts and other low molecular weight species as a way to enrich the HA content of the monovalent strains. Sterile water for injection was used for removal of low molecular weight materials by diafiltration.

To effectively remove surfactant, such as Triton X-100, present in the monovalent bulks, an additional TFF washing step was employed. This washing step consisted of diafiltration prior to concentration using ¼-10 diavolumes of sterile water for injection. Following diafiltration and washing, the volume of each vaccine solution was reduced to 1/20th -1/50th of the original volume, increasing the HA concentration to 5-10 mg HA/mL. This was the concentration limit that the vaccine could be effectively concentrated to by TFF concentration. Further concentration of the monovalent strains was not possible by TFF due an increase in back-pressure most likely caused by fouling of the membrane from insoluble particles in solution (see discussion section). Recovery of the HA concentrate from the TFF system was high, typically >95% as determined by BCA protein assay and SRID potency before and after concentration. Following TFF concentration, the monovalent strains were collected, formulated and then lyophilized as a means to further increase the HA concentration.

### Lyophilization

For pre-clinical studies, following TFF concentration, the monovalent strains were filled into 20 mL glass vials, flash frozen with liquid nitrogen, and placed on a manifold-style freeze drier (Virtis, 25EL Freezemobile). The solutions were allowed to freeze-dry for 2-5 days until the chamber pressure reached a steady state (˜50 mTorr). For clinical production of Phase I materials, 5 mL of the formulated TFF concentrate was filled into 20 mL glass vials and lyophilized in a Stoppering Tray Dryer (Labconco, FreezeZone). Recovery following lyophilization was also high (>90%) as determined by BCA protein assay and SRID of the reconstituted freeze-dried powder.

### HA Purity Determination

HA purity for the monovalent bulk vaccines was determined relative to the total protein and the total solids present in solution. Total protein for the monovalent bulk was measured with the Lowry Assay using Bovine Serum Albumin as reference standard. The % HA purity relative to total protein was then calculated by dividing the known HA content of the sample by the total protein measured. The % HA purity relative to total solids was determined by evaporating a portion of the monovalent bulk to dryness, to determine the total weight of solid present in solution, and dividing this value into the known HA content of the sample.

To estimate the % HA purity in the solid following purification by TFF, a 10 mL aliquot of the monovalent bulk was concentrated in a filtration apparatus (Centricon, Millipore) approximately 10 fold. The concentrate was then washed and re-concentrated with two 10 mL volumes of purified water to remove residual process salts and other low molecular weight materials present in the raw material. The concentrate was then evaporated to dryness and the dry weight of the remaining solid was divided into the amount of HA present in the sample.

The % HA purity was reassessed following the lyophilization process by weighing a portion of the freeze-dried powder and analyzing by SRID following reconstitution with purified water.

### Microprojection Arrays and Coating

Titanium microprojection arrays were fabricated by a photo/chemical etching and formed using a controlled manufacturing process. See, e.g., EP0914178B1.

Coating was conducted at ambient temperature utilizing a roller drum, rotating at 50 rpm, in a drug formulation reservoir (2 mL in volume) to produce a thin film of controlled thickness of ˜100 micrometer in thickness. Microprojection arrays are dipped into the thin film, and the amount of coating is controlled by the number of dips (passes) through the drug film. The time between each dip is approximately 5 seconds, which is sufficient to dry the coated liquid formulation under the ambient condition.

### RESULTS AND DISCUSSION

### Formulation Parameters for Coating

This novel transdermal microprojection patch system featured a solid-state formulation coated on the microprojection array. Therefore, the development of a liquid formulation that enabled the coating process was the precursor to a stable, performance enhancing solid-state formulation.

A liquid formulation was prepared to primarily satisfy three key coating formulation parameters-vaccine concentration, viscosity, and surface activity. More specifically, a liquid formulation with a high vaccine concentration and of sufficiently high viscosity advantageous (but not necessarily required) to ensure that each dip of microprojections into the liquid formulation can pick up sufficient volume of liquid for drying, which can achieve the desired vaccine dose with a minimum number of dips. The viscosity of the coating solution has to be high enough so that the coated liquid will not quickly drip back after dipping but before drying. Also important is the Newtonian behavior of the liquid formulation in the drug coating reservoir, i.e., constant viscosity over shear rate, because the coating process involves a certain level of shear with the roller drum. Surface activity is an aspect to establish a hydrophilic interface between the liquid formulation and the titanium surface, which can be quantified by contact angle measurement. The preferred contact angle is 30 degrees to 60 degrees (referenced to the contact angle of 70 degrees to 80 degrees between water and the titanium surface). A surfactant is often needed if the vaccine formulation is not sufficiently hydrophilic to the titanium surface.

Furthermore, enhancing the vaccine (antigen) purity, i.e., reducing the amount of non-immunogenicity contributing compounds in the formulation, is an important consideration because the formulation is coated at the microprojection head which has limited surface area. Excessive formulation deposited at the microprojection head may blunt the microprojection to hinder skin penetration. The design variables above directed the pre-formulation/formulation approaches as described below.

### Monovalent Bulk

Each bulk solution was turbid as received, suggesting the presence of insoluble particles due possibly to water-insoluble lipids, lipid-protein complexes, and aggregated proteins. The haemagglutinin antigen (HA) concentration in the bulk solution was low, ˜0.1-0.2 mg/mL, and the HA purity was variable, typically in the range of 20±5% of the total solids (low molecular weight solutes, proteins, and insoluble particles) and of 40±10% in total protein content (HA and non-HA proteins). To coat the HA antigen on the microprojection array, the bulk solution needs to be reformulated to increase HA concentration and purity. Since the non-HA proteins and particles may contribute to immunological responses, the HA purity can only be improved by removing the low molecular weight materials, which include buffers, salts and surfactants such as Triton-X 100 (used for splitting the virus particles during vaccine manufacturing). The removal of the low molecular weight species was accomplished by diafiltration.

### Tangential-Flow Filtration (TFF) Process

In the TFF system containing a 30 kD membrane, the monovalent bulk was initially concentrated to reduce its volume to 1/20th- 1/50th of the original volume and washed by 10 diavolumes of 10 mM phosphate buffer. However, this process resulted in a marginal increase in % HA purity, by <15%, and a significant increase in the concentration of Triton-X 100 (MW of 625 Dalton). The formation of higher-molecular weight Triton-X 100 micelles is the reason why this process failed to effectively remove Triton-X 100.

Triton-X 100 is known to form micelles of 80,000 Dalton in molecular weight at the critical micelle concentration (CMC) of 0.13-0.56 mg/mL. The monovalent bulk typically contained 0.1-0.3 mg/mL of Triton-X 100, which is higher than the HA concentration and already close to or reaching its CMC. The initial concentration step thus pushed Triton-X 100 well beyond its CMC to reach a concentration as high as 15 mg/mL and formed Triton-X 100 micelles that were too large to pass through the 30-kD membrane.

Therefore, the process was modified to add an additional wash step prior to concentration. This process maintained a relatively low concentration of Triton-X 100 during diafiltration and allowed for effective reduction of the surfactant from the monovalent bulk. It was found that about 95% of the Triton-X 100 could be removed following two diavolumes of distilled water. Unfortunately, this low level of surfactant worsened the recovery of HA by 5-10%. The mechanism of deteriorating HA recovery is not clear but may be due possibly to decreased solubility of the protein and/or increased hydrophobicity of the diafiltration membrane. The optimal weight ratio of HA to Triton-X 100 was determined to be in the range of 2:1-5:1, which cleared most of the excess Triton-X 100 without compromising the recovery of HA significantly. Following the initial wash, the washed solution was then concentrated down to 1/20th- 1/50th of its original volume, increasing the HA concentration to 5-10 mg HA/mL. The solid composition of the resulting TFF concentrate contained 4±5% HA at (10-15 mg HA/mL concentration); 15±5% Triton-X 100 (3-5 mg/mL), and residual non-HA proteins and insoluble particles making up the remaining weight fraction, 40±10%, of the white turbid solution.

This solution did not reach the target HA concentration of 40-50 mg/mL for coating. Unfortunately, further concentration in the TFF system reached a viscosity limit at which point the fore- and back-pressure became so high that it might jeopardize the integrity of the membrane. Therefore, further concentration was achieved by lyophilization of the TFF concentrate and subsequent reconstitution to a desired HA concentration.

### Lyophilization Process

Prior to freeze-drying, sucrose or trehalose was added to the TFF concentrate as a lyoprotectant (1:1 lyoprotectant:HA weight ratio). The effect of these two disaccharide stabilizers was evaluated by subjecting the formulation to 10 cycles of freeze/thaw (frozen by liquid nitrogen and immediately thawed at room temperature). As determined by ELISA, the HA potency before and after 10 cycles of freeze/thaw was unchanged (data not shown), suggesting the preservation of antigen stability by the trehalose or sucrose. While higher weight ratios of the lyoprotectant are normally required in the solid-state biopharmaceutical formulations to provide long-term stability to the protein, it is more important to limit the total solid content of the formulation in order to keep the coated morphology compact in size on the tips of the microprojections, which is important to penetration efficiency of the microprojection tips. Hereafter, sucrose was added to the TFF freeze concentrate at a 1:1 sucrose:HA weight ratio for lyophilization. The solid composition of the resulting lyophilized vaccine contained 30+5% HA, 30+5% sucrose, 10+5% Triton-X 100, and non-HA related proteins and solid particles making up the remaining fraction of 30+15%.

### Coating Formulation

To prepare the liquid coating solution, each of the lyophilized monovalent formulations were reconstituted with four to five folds less sterile water for injection than the original pre-lyophilized volume to further increase the HA concentration to 40-50 mg HA/mL. This resulted in a fine suspension of the reconstituted vaccine. Aliquots of the reconstituted monovalent solutions were then combined in a 1:1:1 HA ratio based on their SRID potency value to produce the trivalent coating solution, with each strain present at a concentration of ˜14-15 mg HA/mL. Again, the trivalent liquid coating formulation was prepared to satisfy three key coating formulation parameters-vaccine concentration, viscosity, and surface activity.

### Vaccine Concentration

The vaccine concentration of the coating solution was formulated to be as high as possible to minimize the number of coating passes, i.e., the number of dips into the film on the rotating drum, required to achieve the target dose in an effort to minimize the manufacturing time required to produce each patch. However, the viscosity and stability of the coating solution limited vaccine concentration used for coating. In this Example, coating solutions with an HA concentration 60 mg HA/mL or higher were found to be too viscous to form a continuous thin film on the drum and congealed over time under the continuous sheer in the coater. For this reason, the concentration of HA was maintained between 40 and 50 mg total HA/mL. The following table summarizes the HA concentration and purity relative to total solids through the different stages of the pre-formulation/formulation process.

TABLE 1
Summary of HA concentration and purity through the pre-formulation process.

| | | | | Trivalent |
|---|---|---|---|---|
| | Monovalent | TFF | Lyophilized | Coating |
| | Bulk | Concentrate | Powder | Solution |
| HA Concentration | 0.1-0.2 | 5-10 | — | 40-50 total |
| (mg/mL) | | | | (13-17 per |
| | | | | strain) |
| HA purity relative to | 20-30 | 40-50 | 20-30* | 20-30 |
| total solids (%) | | | | |

*Following addition of lyoprotectant

### Viscosity

The viscosity of the coating solution, controlled by the overall concentration of the antigen, the non-HA proteins/particles, and Triton-X 100, affects the flow of the thin film on the microprojection tips during the coating process. Each dip of the microprojection tip can pick up some coating solution. If the solution viscosity is too low, the solution on the microprojection tip may drip back to the coating solution film before it gets dried. If the viscosity of the solution is too high, the liquid will flow too slowly to uniformly coat the microprojections as needed. It was determined experimentally that the viscosity range of the liquid formulation is from 0.20 to 1.50 poise to attain acceptable coating morphology. The viscosity at various shear rates for a flu vaccine formulation of three HA/sucrose (1:1 weight ratio) concentrations, 50, 40, and 35 mg/mL, is depicted in FIG. 2. As expected, the viscosity of the coating solution was found to be directly related to the concentration of HA in the formulation. At the 50 mg/mL HA concentration, the coating solution demonstrated the desired viscosity for coating during the entire range of shear rate and it also required a minimum number of coating passes due to its sufficiently high concentration.

### Surface Activity

The coating solution should also exhibit proper surface activity to effectively wet the microprojection tips. Wettability, depending on the surface tension of the liquid and the surface energy of the substrate, measures the ability of the coating solution to attach, adhere and spread over the surface of the microprojections and can be determined by contact angle measurement. Poor wettability will either discourage fluid uptake or result in uneven, localized coating. Liquid formulation containing surface active agents can affect the surface tension and improve surface wettability by decreasing the contact angle between the solution and the substrate. Compared to the contact angle of pure water on the titanium substrate (80°), the coating formulation (equal weight ratio of HA and sucrose) showed good wettability with contact angles ranging from 26° to 36° regardless of the HA concentration. The HA antigen and/or Triton-X 100 might be the surface active agents in the formulation. In addition, when several surfactants, such as Tween 80, Pluronic F68, and Zwittergent 3-14, were added to the coating formulation (up to 1%), the contact angle on the titanium surface remained the same (data not shown). This observation again suggests that the coating formulation is inherently surface active, which would favor the coating process.

Titanium metal is known to form thin oxide films (primarily TiO2) on the surface, and its surface activity is dynamic depending the thickness, microstructure, and composition of the thin film. Surface absorption of organic compounds from the ambient air also affects surface activity, hydrophilicity or hydrophobicity, significantly. To assess the effect of the surface energy of the titanium metal on wettability of the formulation, titanium metal was pre-treated by heating at 250° C. for 1 hour. High temperature heating can burn off contaminants and shift the surface toward a higher degree of hydrophilicity. Indeed, the pre-heated titanium showed a significant decrease in contact angle of pure water, 50°, compared to 80° on the untreated titanium surface, suggesting a substantial increase in the hydrophilicity (or surface energy) of pre-heated titanium surface. Interestingly, the contact angles of the coating formulation on pre-heated titanium surfaces remained unchanged (26° to 36°), suggesting that the coating formulation overpowered the surface activity of the titanium substrate. Overall, the coating solution exhibited robust wetting properties, which were minimally affected by the coating substrate, and showed excellent coating properties.

### Physical Stability of Coating Formulation

Despite its proper physical properties for coating, the 50 mg/mL HA/sucrose coating formulation is, however, a milky white suspension solution. With no visible particles, this fine suspension may contain mostly nanoparticles. This suspension solution was considered physically stable because there was no phase separation (particle settlement) observed after the solution was kept under refrigeration for a month. Furthermore, there was no clear particle sedimentation after the solution was centrifuged for 2 minutes at 7,000 rpm. Like a stable emulsion, oil-in-water or water-in-oil, which is typically stabilized by an emulsifier (or surfactant), the suspension of nanoparticles is possibly stabilized by Triton-X 100.

### The Coating Process

The coating apparatus comprised a coating solution reservoir and a stainless steel drum which was in contact with the coating solution. The drum was rotated to generate a continuous thin film (˜100 μm thick) of the coating formulation into which the microprojection tips on the titanium arrays was dipped. With precise control of dip depth, only the tips of the microprojections were coated with the coating formulation. Due to the relatively small volume of formulation coated on the tips of the microprojections, the high solid content of the formulation, and the very high surface area of the array, the liquid coating on the microprojection surface is expected to be air dried in less than 5 seconds after coating under ambient conditions. The coated amount of vaccine was controlled by the number of times the array was dipped into the thin film and was monitored by BCA and/or SRID.

FIG. 3 shows representative coating morphology on the microprojection tip. The coating is uniformly distributed over all microprojections (FIG. 3a) and located on the microprojection tip (FIG. 3b-d for side-view, top-view, and front-view of a single microprojection).

As the coating solution is exposed to high shear forces during the coating process, the formulation must be adequately stable in terms of physical stability of the thin film used for coating and chemical stability of the antigen in solution. Physical stability of the coating formulation was determined by monitoring the solution viscosity under prolonged shear simulated in the Rheometer. Physical instability of the coating formulation exposed to constant shear force has been observed for some biopharmaceutical formulations as evidenced by gel formation and breakdown of the thin film resulting in increased solution viscosity. Chemical stability was monitored periodically over a one hour coating run by the in vitro potency assay, SRID. Both viscosity and SRID potency remained unchanged during the one hour exposure to constant shear.

With the pre-formulation and coating processes being developed for monovalent vaccine bulk, the trivalent vaccine formulation was prepared and is described below.

### Trivalent Flu Vaccine Manufacturing

Three monovalent strains A/New Caledonia (H1NI), A/Panama (H3N2) and B/Shandong, at concentrations ranging from 125 to 500 mcg HA/mL, were used for a Phase I clinical manufacturing of the trivalent transdermal delivery systems. Approximately 2 liters of each monovalent strain of bulk viral extract was diaflitered and then concentrated to 10 mg HA/mL on the TFF apparatus. The concentrated monovalent solutions were then individually formulated with a 1:1 weight raito of HA:sucrose and freeze-dried to powder form. The three freeze-dried powders were then reconstituted and combined to produce a 1:1:1 trivalent coating solution at a concentration of 42 mg HA/mL (14 mg HA/mL for each monovalent strain). This coating solution exhibited acceptable viscosity and wettability to coat the target dose of 30 mcg HA trivalent (i.e., ˜10 μg per monovalent strain) with a minimum number of dips per array. After coating, acceptable systems were packaged in nitrogen-purged heat-sealed foil pouches and stored at 2-8° C. Representative systems were selected from the clinical batch and tested for lot release by the SRID assay. All systems tested met lot release specifications of >8 mcg HA/patch. The averages for 20 systems randomly selected throughout the batch were: 11.0 mcg A/New Caledonia, 13.3 mcg A/Panama and 12.2 mcg B/Shangdong with relative standard deviations within 6%.

### Stability Considerations

It is paramount to maintain the antigen's stability throughout the pre-formulation process (diafiltration/concentration, lyophilization, and reconstitution). Other than all the processing stresses, there is a concern about the effect of the high concentration Triton-X 100 present in the coating formulation on HA's antigenicity as the Triton-X 100 concentration was increased by more than 10 fold, from 0.1-0.3 mg/mL to 3-5 mg/mL (see the TFF Process Section).

To evaluate this effect, SDS-PAGE/Western blot analysis was performed on A/Panama vaccine after a series of pre-formulation steps (FIG. 4) including the freeze-dried vaccine reconstituted without surfactants and with three high concentration surfactants, SDS (at 10%), Triton-X 100 (at 10%), or Zwittergent 3-14 (at 5 and 10%). Under the non-reducing conditions for the Coomassie Blue stained gels (SDS-PAGE gels on the left), it is evident that all bands present in the starting vaccine were also present in the reconstituted samples, suggesting no detectable degradation for any of the formulations evaluated. As the gel was transferred to the membrane for Western Blot analysis (FIG. 4, gels on the right), again, no differences were noticed between the different formulations and the starting monovalent vaccine. A series of bands, reflecting the binding between HA protein and anti-HA antibodies, occurred primarily at high molecular weights. Based on the matched bands and band intensity (relative to the starting vaccine), the HA in formulations that had been freeze-dried and exposed to high concentrations of strong surfactants maintained antigenicity. Under reducing conditions, all formulations show bands similar to that of the starting vaccine on SDS-PAGE gels. Band patterns on the Western Blot gels were also matched well among all formulations.

Long-term stability of the final product was assessed using the systems produced during Phase I clinical manufacturing. Nitrogen-purged heat-sealed foil pouches were placed on stability in humidity controlled chambers at 5 and 25° C. for up to 12 months. HA potency for each strain as determined by SRID was used as the stability indicating assay and was compared to T=0 lot release data for each of the three monovalent strains. The data (FIG. 5), reported as a percentage of initial trivalent potency in FIG. 5, suggest that HA maintained good stability (>85% initial) through 12 months at both 5 and 25° C. indicating the potential room temperature stability of this product.

### Immunogenicity Performance of Coated Flu Vaccine System

Pre-clinical immunogenicity data were obtained from hairless guinea pigs, which have a skin structure similar to humans. The positive immune responses in this efficacy animal model prompted our decision to enter into a Phase 1 human trial with the patch formulation. Administration via the transdermal route (7-8 mcg HA per strain for two patch designs) outperformed that via the intramuscular (IM) route (15 μg HA per strain) in terms of Haemagluttin Inhibition) (HAI) percent seroconversion (Table 2). It suggests that even at 50% less antigen, the immune responses (Day 28 post primary immunization) induced by the patch were equivalent to or outperformed those by IM injection.

TABLE 2
Pre-clinical immunogenicity results summary

| | | | Primary Immunization (day 28) | | |
|---|---|---|---|---|---|
| | | | % seroconversion (n/total) | | |
| | Patch | | HAI (≥10) | | |
| Delivery | Wearing | Trivalent HA | Strain | Strain | |
| Route | Time | dose (1:1:1) | A/H1N1 | A/H3N2 | Strain B |
| IM | Not | 45 μg | 90 (9/10) | 30 (3/10) | 40 (4/10) |
| | applicable | | | | |
| Patch | 15 min | 21 μg | 100 (10/10) | 60 (6/10) | 90 (9/10) |
| Design #1 | | | | | |
| Patch | 15 min | 25 μg | 100 (10/10) | 50 (5/10) | 60 (6/10) |
| Design #2 | | | | | |

### Human Clinical Validation of the Performance and Safety of the Microneedle Device for Vaccine Delivery

A Phase 1 clinical study (single center, open label, randomized) was executed in order to compare the efficacy of trivalent influenza antigens administered by microneedle patch versus delivery of the trivalent vaccine via standard intramuscular route. For this study, healthy men and women (ages 18-40; ˜30 subjects/group) were treated with either a microneedle patch coated with each antigen strain (A/New Caledonia (H₃N₂), A/Panama (H₃N₂), B/Shandong); 12 μg/each) or the commercial IM-delivered vaccine (15 μg of each strain). Once administered, the patch was worn for 5 or 15 minutes.

Results obtained in the immunogenicity analysis set are presented in Table 3 by strain for all three groups.

TABLE 3
Immunogenicity Results (HI − 1/dil) Following EMEA
Criteria Note for Guidance Immunogenicity Analysis Set

| | | | | 15 minute | | | |
|---|---|---|---|---|---|---|---|
| | EMEA | 5 minute patch | | patch wear | | | |
| | requirement for | wear time | | time | | IM | |
| | 18 to 60 years* | N = 32 ¶ | | N = 29 ¶ | | N = 29 ¶ | |
| Immunogenicity | | 27 | | 27 | | 28 | |
| Analysis Set for | | | | | | | |
| all three strains | | | | | | | |
| (n) | | | | | | | |
| Strain: A/H1N1 | | | | | | | |
| Seroconversion | >40% | 81.5 | (61.9-93.7) | 96.3 | (91.0-99.9) | 89.3 | (71.8-97.7) |
| rate¹ or | | | | | | | |
| significant | | | | | | | |
| increase of HI | | | | | | | |
| titer ²at Day 21 | | | | | | | |
| Geometric mean | 2.5 | 18.0 | (10.5-30.7) | 50.1 | (31.3-80.2) | 27.2 | (15.3-48.4) |
| of titers increase\ | | | | | | | |
| Percentage of | >70% | 88.9 | (70.8-97.6) | 100 | (87.2-100) | 92.9 | (76.5-99.1) |
| seroprotected | | | | | | | |
| subjects** at | | | | | | | |
| Day 21 | | | | | | | |
| Strain: A/H3N2 | | | | | | | |
| Seroconversion | >40% | 44.4 | (25.5-64.7) | 51.9 | (31.9-71.3) | 71.4 | (51.3-86.3) |
| rate¹ or | | | | | | | |
| significant | | | | | | | |
| increase of HI | | | | | | | |
| titer² at Day 21 | | | | | | | |
| Geometric mean | 2.5 | 4.91 | (2.61-9.24) | 4.10 | (2.56-6.57) | 8.72 | (4.45-17.1) |
| of titers increase\ | | | | | | | |
| Percentage of | >70% | 100 | (87.2-100) | 96.3 | (81.0-99.9) | 100 | (8.7-100) |
| seroprotected | | | | | | | |
| subjects ** at | | | | | | | |
| Day 21 | | | | | | | |
| Strain: B | | | | | | | |
| Seroconversion | >40% | 74.1 | (53.7-88.9) | 74.1 | (53.7-88.9) | 67.9 | (47.6-84.1) |
| rate¹ or | | | | | | | |
| significant | | | | | | | |
| increase of HI | | | | | | | |
| titer² at Day 21 | | | | | | | |
| Geometric mean | 2.5 | 13.7 | (8.81-21.3) | 13.9 | (8.41-23.0) | 10.4 | (6.15-17.5) |
| of titers increase\ | | | | | | | |
| Percentage of | >70% | 81.5 | (61.9-93.7) | 77.8 | (57.7-91.4) | 75.0 | (55.1-89.3) |
| seroprotected | | | | | | | |
| subjects ** at | | | | | | | |
| Day 21 | | | | | | | |

*EMEA guidance: Committee for proprietary medicinal products (CPMP), note for guidance on harmonization of requirements for influenza vaccines, March 1997;
¹Proportion of subjects with a pre-vaccination titer <10 (1/dil) to a post vaccination titer >=40 (1/dil);
²Proportion of subjects with a pre-vaccination titer <10 to a post vaccination titer >=4-fold titer;
\in the “Guidance”: Mean geometric increase between Day 0 and Day 21;
** Proportion of subjects achieving a post vaccination titer >=40 (1/dil);
¶ Statistic with 95% confidence interval.

The three EMEA criteria were met for all strains 21 days after vaccination, for all three treatment groups. The immunogenicity results of both microneedle patch groups were globally similar to those in the IM group. The patch wear time does not appear to largely affect the degree of antibody response. Total IgE (non-specific) data were similar between the three groups ranging between 24.7 and 41.6 kU/L. For IgA and IgG (against the A/H1N1) strain), values at Day 0 (pre-vaccination) were similar between the groups. 21 days after vaccination both IgA and IgG were 5 to 11 fold higher than at Day 0 and there was no difference between groups. The microneedle patch groups demonstrated a similar immune response to IM control.

### CONCLUSION

A trivalent influenza vaccine transdermal patch was successfully developed and proven to be efficacious pre-clinically and in a Phase I human clinical trial. A unique pre-formulation process that consists of diafiltration/initial concentration by a TFF system, freeze-drying and reconstitution to prepare high HA concentration (˜40-50 mg HA/mL) solutions for coating was established. This pre-formulation process was highly efficient resulting in very little loss of antigen (process yield >85%). The subsequent coating solution prepared following the pre-formulation process was optimized to possess acceptable physical and chemical stability for coating onto the tips of titanium microprojections. The patch formulation demonstrated three key advantages over the currently available formulations: preservative-free, room-temperature storage and dose sparing. Based on the success of the above transdermal patch, a person of ordinary skill in the art will appreciate that such techniques can be extended to any vaccine that can be formulated in a coating solution (using similar or different excipients) and applied to microprojection arrays in therapeutically effective amounts.

### Example 2—Coronavirus Vaccine Employing Synthetic Peptide Antigens on a Transdermal Microprojection Patch

Coronavirus vaccine patches will be prepared generally in accordance with Example 1, except that the antigen will be synthetic peptides. In this non-limiting example, such synthetic peptides will be five peptide antigens. The five peptides are mixed at a 1:1:1:1:1 ratio. The mixture is then coated onto the microprojection patch at a dose of about 50 to about 100 mcg per peptide. The mixture is formulated at about pH 3 to about 9.5. The five peptides are as follows:

HP201-215
DLFGIWSKVYDPLYC
NS3974
YNGSICVIGTPLSRFMGF
Core57
AKRRRRHRRDQGGWRRSP
Core78
VDPYVRQGLQILLPSAAY
Core113
GTLGWTADLLHHVPLVGP

The patches will be assessed by SDS-PAGE/Western Blot, and pre-clinical immunogenicity data will be obtained from hairless guinea pigs generally in accordance with the procedures described in Example 1.

Human clinical trials will also be performed in general accordance with the protocol of Example 1.

Applicant expects that when the vaccine patch of Example 2 is administered to a population of patients, a statistically significant number of such patients will be successfully vaccinated. In other embodiments, at least 10% of such patients will be seroprotected, or at least 20%, or at least 30%, or at least 40%, or at least 50%, or at least 60%, or at least 70%, or at least 80%, or at least 90% of such patients will be seroprotected.

In other aspects, the vaccine coated patches of Example 2 will be dose-sparing as compared with IM or SC injectable vaccine counterparts. For instance, the patches herein will require at least 5%, or at least 10%, or at least 20%, or at least 30% less vaccine/antigen than their IM or SC injectable counterparts.

While the invention has been described in conjunction with specific embodiments thereof, it is to be understood that the foregoing description as well as the examples are intended to illustrate and not limit the scope of the invention. Other aspects, advantages and modifications within the scope of the invention will be apparent to those skilled in the art to which the invention pertains.

We claim:

1. An intracutaneous delivery system comprising a plurality of microprojections that are adapted to penetrate or pierce the stratum corneum of a human patient, the microprojections having a coating thereon comprising a therapeutically effective amount of a vaccine.

2. The system of claim 1 wherein at least 95% of the vaccine is released within about 20 minutes after application of the system to the stratum corneum of the human patient.

3. The system of claim 12, wherein at least 95% of the vaccine is released within about 5 minutes after application of the system to the stratum corneum of the human patient.

4. The system of claim 1 wherein the vaccine is a coronavirus vaccine.

5-6. (canceled)

7. The system of claim 1, wherein the coating further comprises a disaccharide and/or a surfactant.

8. The system of claim 7, wherein the coating comprises a disaccharide is selected from sucrose and trehalose.

9. (canceled)

10. The system of claim 1, wherein the system is stable at room temperature for at least 6 months.

11. The system of claim 10 wherein the system is stable at room temperature for at least 12 months.

12. A method for vaccinating a human patient against a coronavirus or an influenza disease, comprising the steps of:
(a) Providing an intracutaneous delivery system comprising a plurality of microprojections that are adapted to penetrate or pierce the stratum corneum of a human patient, the microprojections having a coating thereon comprising a therapeutically effective amount of a vaccine;
(b) applying the microprojections to a selected area of skin of the patient.

13. The method of claim 12 wherein the disease is COVID-19.

14-15. (canceled)

16. The method of claim 12 wherein the vaccine is a coronavirus vaccine.

17-18. (canceled)

19. The method of claim 12, wherein the coating further comprises a disaccharide and/or a surfactant.

20. The method of claim 19, wherein the coating comprises a disaccharide selected from sucrose and trehalose.

21-23. (canceled)

24. The method of claim 12, wherein the vaccine is self-administered.

25-28. (canceled)

30. The method of claim 12, wherein the system has a wear time of about 5 to 30 minutes.

31. The system of claim 4, wherein the vaccine is against SARS-COV-2.

32. The system of claim 1, wherein the vaccine is an influenza vaccine.

33. The system of claim 1, wherein the vaccine is a peptide vaccine.

34. The system of claim 1, wherein the vaccine, when administered to a subject, stimulates production of antibodies.

35. The system of claim 1, wherein the system comprises a disposable patch assembly having a plurality of microprojections disposed in an array of about 3 cm² to about 6 cm², the array having a density of about 200 to about 2000 microprojections/cm².

36. The system of claim 1, wherein the microprojections are rectangular, with a triangular tip to facilitate penetration of the stratum corneum of a human; and/or
wherein the microprojections have:
(i) a length of about 25 to about 600 μm;
(ii) a width of about 10 μm to about 500 μm;
(iii) a thickness of about 1 μm to about 500 μm; and
(iv) a tip angle of about 30 to about 70 degrees.

37. The system of claim 1, wherein the coating covers about 10% to 80% of the length of each microprojection measured from the tip to the base.

38. The method of claim 12, wherein the system comprises a disposable patch assembly having a plurality of microprojections disposed in an array of about 3 cm² to about 6 cm², the array having a density of about 200 to about 2000 microprojections/cm².

39. The method of claim 12, wherein the microprojections are rectangular, with a triangular tip to facilitate penetration of the stratum corneum of a human; and/or wherein the microprojections have:
(i) a length of about 25 to about 600 μm;
(ii) a width of about 10 μm to about 500 μm;
(iii) a thickness of about 1 μm to about 500 μm; and
(iv) a tip angle of about 30 to about 70 degrees.

40. The method of claim 12, wherein the coating covers about 10% to 80% of the length of each microprojection measured from the tip to the base.

41. The method of claim 12, wherein the vaccine is an influenza vaccine.

42. The system of claim 1, wherein the coating is a solid coating.

43. The system of claim 7, wherein the coating comprises a surfactant, and the surfactant is polysorbate 20.

43. The method of claim 12, wherein the coating is a solid coating.

44. The method of claim 19, wherein the coating comprises a surfactant, and the surfactant is polysorbate 20.


SEQUENCE LISTING (5 sequences)

SEQ ID NO: 1 - protein, 15 aa, Artificial Sequence
  Peptide HP201-215
DLFGIWSKVYDPLYC

SEQ ID NO: 2 - protein, 18 aa, Artificial Sequence
  Peptide NS3974
YNGSICVIGTPLSRFMGF

SEQ ID NO: 3 - protein, 18 aa, Artificial Sequence
  Peptide Core57
AKRRRRHRRDQGGWRRSP

SEQ ID NO: 4 - protein, 18 aa, Artificial Sequence
  Peptide Core78
VDPYVRQGLQILLPSAAY

SEQ ID NO: 5 - protein, 18 aa, Artificial Sequence
  Peptide Core113
GTLGWTADLLHHVPLVGP